(19) United States
(12) Patent Application Publication
(10) Pub. No.: US 2024/0238367 A1
(43) Pub. Date: Jul. 18, 2024

(54) COMPOUNDS FOR TREATING CONDITIONS RELATED TO PCSK9 ACTIVITY

(71) Applicant: MERCK SHARP & DOHME LLC, RAHWAY, NJ (US)

(72) Inventors: Douglas G. JOHNS, Rahway, NJ (US); Puja BANKA, Rahway, NJ (US); Zexun ZHOU, Rahway, NJ (US); Artis KLAPARA, Rahway, NJ (US); Fuh-rong TSAY, Rahway, NJ (US); Jongrock KONG, Princeton, NJ (US); Richard J. VARSOLONA, Scotch Plains, NJ (US); Richard DESMOND, Labanon, NJ (US); Peter E. MALIGRES, Rahway, NJ (US); Melanie MAROTA, West Point, PA (US); Candice ALLEYNE, Rahway, NJ (US); Grace A. OKOH, Rahway, NJ (US); James C. DINUNZIO, Rahway, NJ (US); Rebecca NOFSINGER, West Point, PA (US); Li LI, Rahway, NJ (US); Daniel J. SMITH, Rahway, NJ (US); Majid MAHJOUR, Schwenksville, PA (US); Kenneth Alan KOEPLINGER, Lansdale, PA (US); Yusheng XIONG, Plainsboro, NJ (US); Peter W. WUEFLING, West Point, PA (US); John D. HIGGINS, South San Francisco, CA (US)

(21) Appl. No.: 18/684,095

(22) PCT Filed: Aug. 18, 2022
(86) PCT No.: PCT/US2022/040747

§ 371 Date: Feb. 15, 2024

Related U.S. Application Data
(60) Provisional application No. 63/234,973, filed on Aug. 19, 2021.
(60) Provisional application No. 63/251,972, filed on Oct. 4, 2021.
(60) Provisional application No. 63/263,095, filed on Oct. 27, 2021.
(60) Provisional application No. 63/311,622, filed on Feb. 18, 2022.
(60) Provisional application No. 63/371,685, filed on Aug. 17, 2022.

(51) Int. Cl. A61K 38/12; A61K 9/00; A61K 9/48; A61K 47/12; A61P 3/06

(52) U.S. Cl. CPC A61K 38/12; A61K 9/0053; A61K 9/4825; A61K 9/4858; A61K 9/4866; A61K 47/12; A61P 3/06

(57) ABSTRACT

The present disclosure provides a method of treating hypercholesterolemia and other conditions related to PCSK9 activity, e.g. atherosclerosis, atherosclerotic cardiovascular disease, coronary heart disease, metabolic syndrome, acute coronary syndrome, or related cardiovascular disease and cardiometabolic conditions, by orally administering to the subject an amount of a compound of formula (I) wherein A″ is selected from a pharmaceutically acceptable anion, and wherein the amount administered is from about 5 mg to about 300 mg of the compound of formula (I). The present invention is also directed to pharmaceutical compositions comprising a compound of formula (I), including particular salts of a compound of formula (I), and a permeation enhancer.

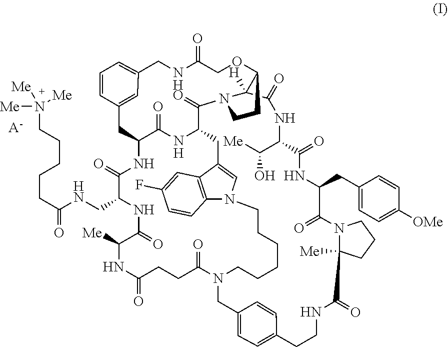

### RELATED APPLICATIONS

This application claims priority to U.S. Provisional Application No. 63/234,973 filed on Aug. 19, 2021, U.S. Provisional Application No. 63/251,972 filed on Oct. 4, 2021, U.S. Provisional Application No. 63/263,095 filed on Oct. 27, 2021, U.S. Provisional Application No. 63/311,622 filed on Feb. 18, 2022, and U.S. Provisional Application No. 63/371,685 filed on Aug. 17, 2022. The contents of each application are hereby incorporated by reference in their entireties.

### FIELD OF THE INVENTION

This disclosure relates to methods of treating hypercholesterolemia and other conditions related to PCSK9 activity, e.g., atherosclerosis, atherosclerotic cardiovascular disease, coronary heart disease, metabolic syndrome, acute coronary syndrome, or related cardiovascular disease and cardiometabolic conditions.

### BACKGROUND OF THE INVENTION

Proprotein convertase subtilisin-kexin type 9 (hereinafter called “PCSK9”), also known as neural apoptosis-regulated convertase 1 (“NARC-1”), is a proteinase K-like subtilase identified as the ninth member of the secretory subtilase family; see Seidah et al., 2003 PNAS 100:928-933. PCSK9 belongs to the mammalian proprotein convertase family of serine proteases and contains an N-terminal signal sequence, a prodomain, a catalytic domain, and a C-terminal domain; see Seidah et al., 2012 Nat. Rev. Drug Discov. 11:367-383. A study of PCSK9 transcriptional regulation demonstrated that it is regulated by sterol regulatory element-binding proteins (“SREBP”), as seen with other genes involved in cholesterol metabolism; Maxwell et al., 2003 J. Lipid Res. 44:2109-2119, as is typical of other genes implicated in lipoprotein metabolism; Dubuc et al., 2004 Arterioscler. Thromb. Vasc. Biol. 24:1454-1459. Statins have been shown to upregulate PCSK9 expression in a manner attributed to the cholesterol-lowering effects of the drugs; supra. Moreover, it has been shown that PCSK9 promoters possess two conserved sites involved in cholesterol regulation, a sterol regulatory element and an Sp1 site; supra.

While in the endoplasmic reticulum, PCSK9 performs as its only catalytic activity an autocleavage between residues Gln-152 and Ser-153; see Naureckiene et al., 2003 Arch. Biochem. Biophys. 420:55-67; Seidah et al., 2003 Proc. Natl. Acad. Sci. U.S.A. 100:928-933. The prodomain remains tightly associated with the catalytic domain during subsequent trafficking through the trans-Golgi network. The maturation via autocleavage has been demonstrated to be critical for PCSK9 secretion and subsequent extracellular function (see Benjannet et al., 2012 J. Biol. Chem. 287:33745-33755). Accordingly, several lines of evidence demonstrate that PCSK9, in particular, lowers the amount of hepatic LDLR protein and thus compromises the liver's ability to remove low density lipoprotein (“LDL”) cholesterol from the circulation.

Adenovirus-mediated overexpression of PCSK9 in the liver of mice results in the accumulation of circulating low density lipoprotein cholesterol (“LDL-C”) due to a dramatic loss of hepatic LDLR protein, with no effect on LDLR mRNA levels; Benjannet et al., 2004 J. Biol. Chem. 279:48865-48875; Maxwell & Breslow, 2004 PNAS 101:7100-7105; Park et al., 2004 J. Biol. Chem. 279:50630-50638; and Lalanne et al., 2005 J. Lipid Res. 46:1312-1319. The effect of PCSK9 overexpression on raising circulating LDL-C levels in mice is completely dependent on the expression of LDLR, again indicating that the regulation of LDL-C by PCSK9 is mediated through downregulation of LDLR protein. In agreement with these findings, mice lacking PCSK9 or in which PCSK9 mRNA has been lowered by antisense oligonucleotide inhibitors have higher levels of hepatic LDLR protein and a greater ability to clear circulating LDL-C; Rashid et al., 2005 PNAS 102:5374-5379; and Graham et al., 2007 J. Lipid Res. 48(4):763-767. In addition, lowering PCSK9 levels in cultured human hepatocytes by siRNA also results in higher LDLR protein levels and an increased ability to take up LDL-C; Benjannet et al., 2004 J. Biol. Chem. 279:48865-48875; and Lalanne et al., 2005 J. Lipid Res. 46: 1312-1319. Together, these data indicate that PCSK9 action leads to increased LDL-C by lowering LDLR protein levels.

A number of mutations in the gene PCSK9 have also been conclusively associated with autosomal dominant hypercholesterolemia (“ADH”), an inherited metabolism disorder characterized by marked elevations of low density lipoprotein (“LDL”) particles in the plasma, which can lead to premature cardiovascular failure; see Abifadel et al., 2003 Nature Genetics 34:154-156; Timms et al., 2004 Hum. Genet. 114:349-353; Leren, 2004 Clin. Genet. 65:419-422. A later-published study on the S127R mutation of Abifadel et al., supra, reported that patients carrying such a mutation exhibited higher total cholesterol and apoB100 in the plasma attributed to (1) an overproduction of apoB100-containing lipoproteins, such as low density lipoprotein (“LDL”), very low density lipoprotein (“VLDL”) and intermediate density lipoprotein (“IDL”), and (2) an associated reduction in clearance or conversion of said lipoproteins; Ouguerram et al., 2004 Arterioscler. Thromb. Vasc. Biol. 24:1448-1453.

Accordingly, there can be no doubt that PCSK9 plays a role in the regulation of LDL. The expression or upregulation of PCSK9 is associated with increased plasma levels of LDL cholesterol, and the corresponding inhibition or lack of expression of PCSK9 is associated with reduced LDL cholesterol plasma levels. Decreased levels of LDL cholesterol associated with sequence variations in PCSK9 have been found to confer protection against coronary heart disease; Cohen, 2006 N. Engl. J. Med. 354:1264-1272.

In clinical trials, reductions in LDL cholesterol levels have been directly related to the rate of coronary events; Law et al., 2003 BMJ 326:1423-1427. The moderate lifelong reduction in plasma LDL cholesterol levels was found to correlate with a substantial reduction in the incidence of coronary events; Cohen et al., 2006 N. Engl. J. Med. 354:1264-1272. This was the case even in populations with a high prevalence of non-lipid-related cardiovascular risk factors; supra. Accordingly, there is great benefit to be reaped from the managed control of LDL cholesterol levels.

Thus, identification of compounds and/or agents effective in the treatment of cardiovascular affliction is highly desirable, including antagonism of PCSK9′s role in LDL regulation; however, in general, because PCSK9 circulates in blood and has modest binding affinity to cell surface LDL receptors heretofore attempts to utilize this mechanism in treatment of diseases related to high serum LDL levels have been focused on the use of large biomolecules, for example, antibodies. Although either PCSK9-directed siRNA or monoclonal antibodies (mAb) therapy can reduce LDL-C in patients with hypercholesterolemia, both types of therapy are dosed by injection. The therapeutic potential of small peptides or small molecules as drugs targeting PCSK9 has only just begun to be explored; see for example, Tombling et al., Atherosclerosis 330 (2021) 52-60. Moreover, there is a paucity of compounds which are amenable to formulation into a dosage form for utilizing an oral administration route of dosing such compounds, a route which would be highly desirable for the provision of therapy for conditions in which regulation of the activities of PCSK9 could play a role.

WO 2019/246349 discloses cyclic peptide compounds useful in the treatment of cardiovascular disease and conditions related to PCSK9 activity. The present disclosure advances the state of the art by providing methods of treating hypercholesterolemia and other conditions related to PCSK9 activity desirably comprising oral administration of an identified PCSK9 inhibitor. Also provided herein are new salt forms of a PCSK9 inhibitor.

### SUMMARY OF THE DISCLOSURE

The present disclosure provides a method of treating hypercholesterolemia and other conditions related to PCSK9 activity, e.g. atherosclerosis, atherosclerotic cardiovascular disease, coronary heart disease, metabolic syndrome, acute coronary syndrome, or related cardiovascular disease and cardiometabolic conditions, comprising orally administering to a subject in need an amount of a compound of formula (I)

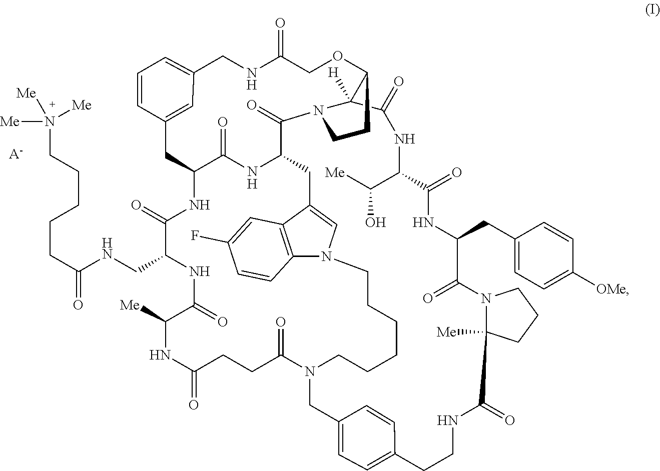

wherein A⁻ is selected from a pharmaceutically acceptable anion, and wherein the amount administered is from about 5 mg to about 300 mg of the compound of formula (I).

The present disclosure also provides a method of reducing LDL-C in a subject in need thereof comprising orally administering to the subject an amount of a compound of formula (I), wherein A⁻ is selected from a pharmaceutically acceptable anion, and wherein the amount is from about 5 mg to about 300 mg of the compound of formula (I).

The present disclosure provides a method of treating atherosclerotic cardiovascular disease in a subject in need of such treatment comprising orally administering to the subject an amount of a compound of formula (I), wherein A⁻ is selected from a pharmaceutically acceptable anion, and wherein the amount administered is from about 5 mg to about 300 mg of the compound of formula (I).

The present disclosure also provides a method of inhibiting PCSK9 activity in a subject in need thereof comprising orally administering to the subject an amount of a compound of formula (I), wherein A⁻ is selected from a pharmaceutically acceptable anion, and wherein the amount is from about 5 mg to about 300 mg of the compound of formula (I).

The present invention is also directed to particular salts of the compound of formula (I).

The present invention is also directed to pharmaceutical compositions comprising a compound of formula (I), including particular salts of the compound of formula (I), and a permeation enhancer.

### BRIEF DESCRIPTION OF THE FIGURE

FIG. 1 shows the pharmacokinetics of single doses of Compound 1, a compound of formula (I), in varying doses from about 10 to about 300 mg.

FIG. 2 shows a summary of the statistics of plasma pharmacokinetics following administration of single oral doses of 10 to 300 mg of Compound 1 to healthy male participants.

FIG. 3 shows the % change from baseline LDL-C for formulations of the compound of formula (I), as well as known anti-PCSK9 monoclonal antibodies and an anti-PCSK9 siRNA active, and placebo.

FIG. 4 shows the reduction of plasma levels of free PCSK9, compared to baseline, after single doses of Compound 1.

FIG. 5 depicts the stability differences between Compound 1, Compound 2 and Compound 3.

### DETAILED DESCRIPTION OF THE INVENTION

This disclosure relates to methods of treating hypercholesterolemia and other conditions related to PCSK9 activity, e.g. atherosclerosis, atherosclerotic cardiovascular disease, coronary heart disease, metabolic syndrome, acute coronary syndrome, or related cardiovascular disease and cardiometabolic conditions. According to the methods of the disclosure, a compound of formula (I)

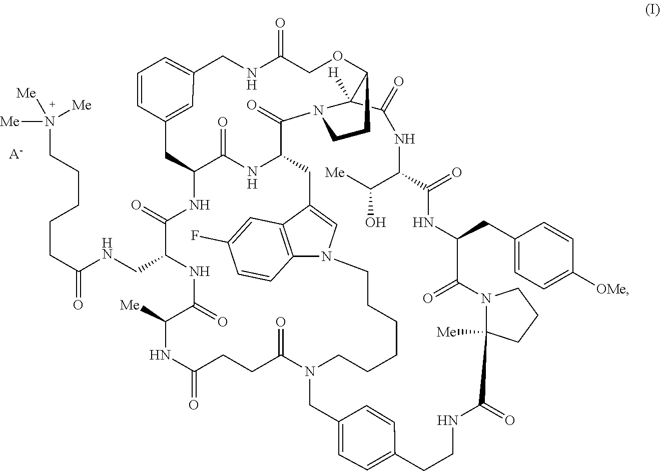

where A⁻ is selected from a pharmaceutically acceptable anion, is orally administered to a subject in need of treatment.

In one embodiment, the present disclosure provides a method of treating hypercholesterolemia in a subject in need of such treatment, comprising orally administering to the subject an amount of a compound of formula (I), wherein A⁻ is selected from a pharmaceutically acceptable anion, and wherein the amount is from about 5 mg to about 300 mg of the compound of formula (I).

In an embodiment, the disclosure is directed generally to a method of reducing LDL-C in a subject in need thereof comprising orally administering to the subject an amount of a compound of formula (I), wherein A⁻ is selected from a pharmaceutically acceptable anion, and wherein the amount is from about 5 mg to about 300 mg of the compound of formula (I).

In an embodiment, the disclosure is directed to a method of treating atherosclerotic cardiovascular disease in a subject in need of such treatment comprising orally administering to the subject an amount of a compound of formula (I), wherein A⁻ is selected from a pharmaceutically acceptable anion, and wherein the amount is from about 5 mg to about 300 mg of the compound of formula (I).

In an embodiment, the disclosure is directed generally to a method of inhibiting PCSK9 activity in a subject in need thereof comprising orally administering to the subject an amount of a compound of formula (I), wherein A⁻ is selected from a pharmaceutically acceptable anion, and wherein the amount is from about 5 mg to about 300 mg of the compound of formula (I). As used herein, “inhibiting” or “antagonizing” refers to providing to affected tissue(s) the compound of formula (I) which opposes the action of, inhibits, counteracts, neutralizes or curtails one or more activities or functions of PCSK9 in the affected tissue(s). In some embodiments, the methods for inhibiting PCSK9 activity are for the treatment of a condition related to PCSK9 activity, as noted above, or, alternatively, for providing therapy in a disease, disorder or condition that could benefit from the effects of a PCSK9 antagonist.

The following details regarding the compound of formula (I), its pharmaceutically acceptable anions, the amount thereof, the subject treated, oral administration, oral dosage forms, formulations, pharmaceutically acceptable excipients, LDL-C reduction, PCSK9 inhibition, etc. relate to all of the methods of the disclosure noted above and below.

The compound of formula (I)

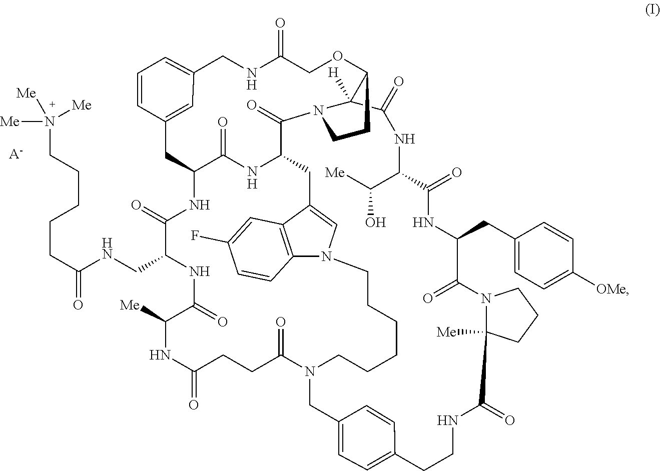

also referred to as “Compound A,” where A⁻ is selected from a pharmaceutically acceptable anion, is used in all of the methods of the disclosure. As used herein, “pharmaceutically acceptable anion” refers to an anion suitable for forming a pharmaceutically acceptable salt.

The term “salt(s)” and its use in the phrase “pharmaceutically acceptable salts” employed herein, includes any of the following: acidic salts formed with inorganic and/or organic acids, basic salts formed with inorganic and/or organic bases, zwitterionic and quaternary ammonium complexes. Salts of compounds of the invention may be formed by methods known to those of ordinary skill in the art, for example, by reacting a compound of the invention with an amount of acid or base, such as an equivalent amount, in a medium such as one in which the salt precipitates or in aqueous medium followed by lyophilization.

Compounds of the invention contain tetra-coordinate positively charged nitrogen atoms, which can be stabilized by the addition of an anion to form a salt or via formation of an anion in a different part of the molecule to generate a zwitterion, sometimes referred to as an inner salt. Accordingly, compounds of the invention may be prepared in the form of a quaternary ammonium salt or quaternary ammonium zwitterion.

Accordingly, structural representation of compounds of the invention, whether in a salt form or a zwitterionic form, also include all other forms of such compounds discussed above. Thus, one aspect of the invention is the provision of compounds of the invention in the form of a pharmaceutically acceptable salt or zwitterion. Those skilled in the art will recognize those instances in which the compounds of the invention may form such salts, including where a tetracoordinate nitrogen can be quaternized and the charged nitrogen form stabilized by an associated anion. The term “pharmaceutically acceptable salt” refers to any salt (including a salt and an inner salt such as a zwitterion) which is not biologically or otherwise undesirable (e.g., is neither toxic nor otherwise deleterious to the recipient thereof).

The formation of pharmaceutically acceptable salts from basic (or acidic) pharmaceutical compounds are generally discussed, for example, by S. Berge et al., Journal of Pharmaceutical Sciences (1977) 66 (1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference.

The present disclosure contemplates all available salts as the compound of formula (I), including salts which are generally recognized as safe for use in preparing pharmaceutical formulations and those which may be formed presently within the ordinary skill in the art and are later classified as being “generally recognized as safe” for use in the preparation of pharmaceutical formulations, termed herein as “pharmaceutically acceptable salts”.

Examples of pharmaceutically acceptable acid salts include, but are not limited to, acetates, including trifluoroacetate salts, adipates, alginates, ascorbates, aspartates, benzoates, benzenesulfonates, bisulfates, borates, butyrates, citrates, camphorates, camphorsulfonates, caprates (also known as decanoates), cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates), undecanoates, and the like. Accordingly, the pharmaceutically acceptable anion corresponding to each of these salts could be A⁻, according to the present disclosure.

Further examples of pharmaceutically acceptable salts, and their corresponding pharmaceutically acceptable anions, that may be used with the present disclosure include, but are not limited to, fluoride, chloride, bromide, iodide, acetate and caprate. In an embodiment of the instant invention, the pharmaceutically acceptable anion is selected from chloride, acetate or caprate. As used herein, caprate and decanoate are used interchangeably.

In an embodiment of the instant invention, the pharmaceutically acceptable anion is chloride, and the compound of formula (I) is

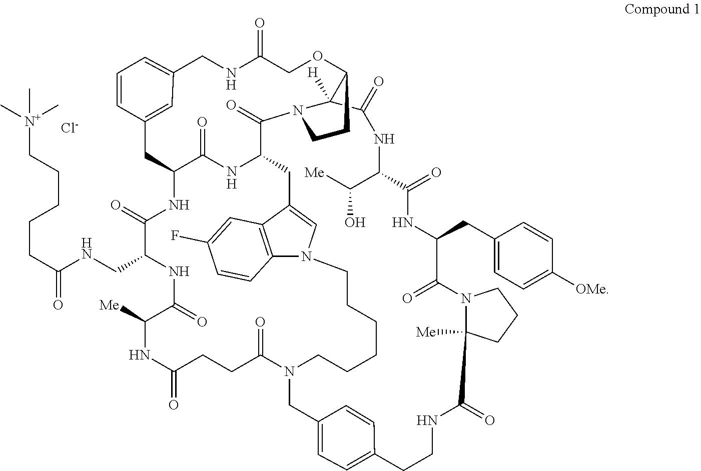

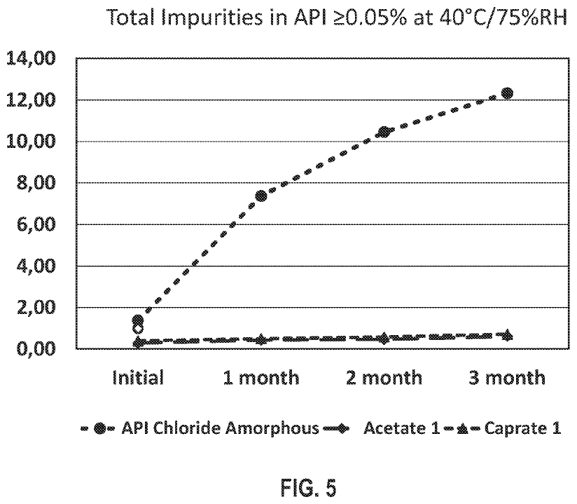

In an embodiment, Compound 1 is in an amorphous form of the chloride salt of a compound of formula (I). In FIG. 5, “API Amorphous Chloride” refers to Compound 1.

In an embodiment of the instant invention, the pharmaceutically acceptable anion is caprate, and the compound of formula (I) is

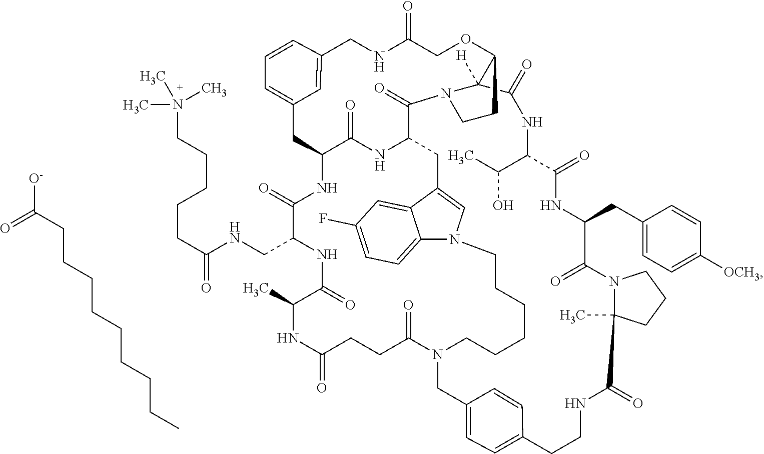

referred to herein as “Compound 2.” In an embodiment, Compound 2 is in an amorphous form of the caprate salt of the compound of formula (I).

In an embodiment of the instant invention, the pharmaceutically acceptable anion is acetate, and the compound of formula (I) is

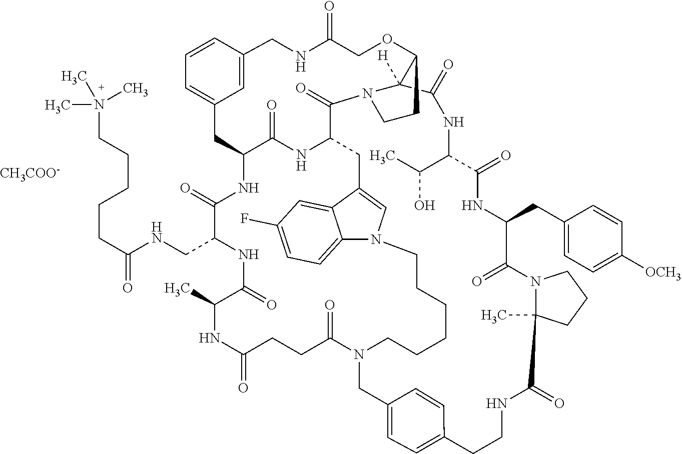

referred to herein as “Compound 3.” In an embodiment, Compound 3 is in an amorphous form of the acetate salt of the compound of formula (I).

In an embodiment, a method of the instant invention comprises administering a compound of formula (I), wherein the compound of formula (I) is selected from

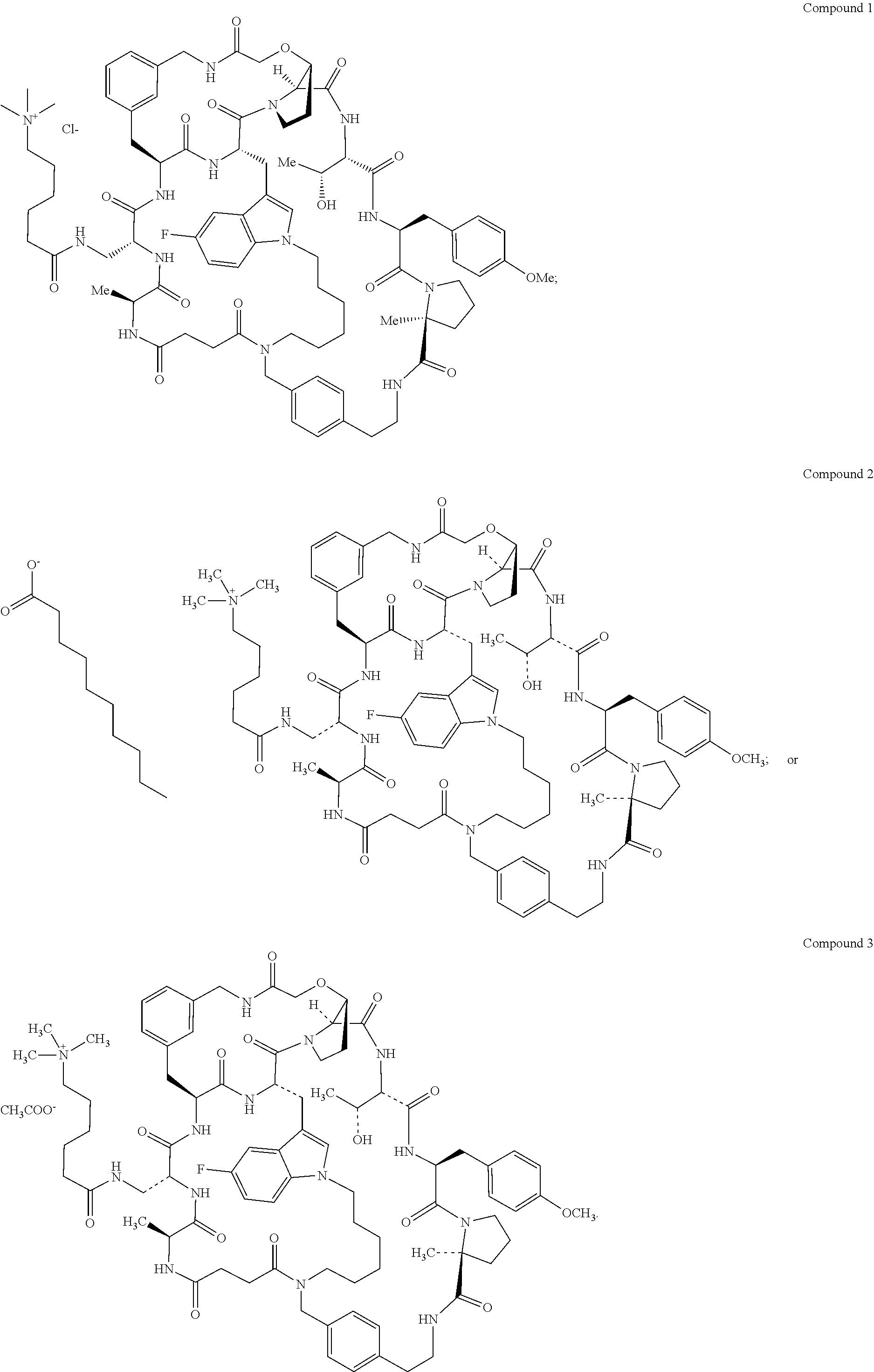

The preparation of the amorphous chloride salt (Compound 1) utilizes an acidification step whereby chloride is introduced through addition of hydrochloric acid to the product after supercritical fluid chromatography and evaporation. FIG. 5 demonstrates the risk associated with the addition of excess HCl in a given batch of Compound 1 (referred to as “API Amorphous Chloride”), since this excess hydrochloric acid, which is a strong acid, can produce increased chemical degradation of the molecule, as observed by the higher impurity levels shown for the amorphous chloride salt than either the amorphous acetate or amorphous caprate salts in FIG. 5. While the stability of the amorphous chloride salt is dependent on the process used to synthesize that salt, the amorphous acetate and amorphous caprate are not subject to this risk and demonstrate stability, regardless of the synthetic process utilized.

The present disclosure also provides a method of inhibiting PCSK9 activity in a subject in need thereof comprising orally administering to the subject an amount of a compound of formula (I), wherein A⁻ is selected from a pharmaceutically acceptable anion, and wherein the amount is from about 5 mg to about 300 mg of the compound of formula (I).

The compound of formula (I) is a compound which has properties that antagonize PCSK9 function and is thus a PCSK9-specific antagonist or inhibitor. The compound of formula (I), as well as methods of making the compound, is disclosed in WO 2019/246349 A1, the entire disclosure of which is incorporated by reference herein.

The compound of formula (I) is represented using conventional stereochemical notation for some asymmetric carbon centers. Accordingly, solid black “wedge” bonds represent bonds projecting from the plane of the reproduction medium, while “hashed wedge” bonds represent descending bonds into the plane of the reproduction medium. As is conventional, plain solid lines represent all spatial configurations for the depicted bonding. Accordingly, where no specific stereochemical notation is supplied, the representation contemplates all stereochemical and spatial orientations of the structural features.

The compound of formula (I) is stable. As used herein, “stable” refers to a compound which can be prepared and isolated and whose structure and properties remain or can be caused to remain essentially unchanged for a period of time sufficient to allow use of the compound for the purposes described herein (e.g., therapeutic administration to a subject).

The compound of formula (I) is bioavailable, and particularly is orally bioavailable. As used herein “bioavailable” refers to the ability of the compound of formula (I) to be absorbed and used by the body. As used herein “orally bioavailable” means that the compound of formula (I), when taken by mouth, can be absorbed and used by the body.

As used herein, the term “treating” or “treatment” refers to inhibiting or ameliorating a disease, condition or disorder in a subject who is experiencing or displaying the pathology or symptoms of the disease, condition or disorder. For example, inhibiting a disease, condition, or disorder refers to arresting further development of the pathology and/or symptoms of said disease, condition or disorder. Additionally, ameliorating a disease, condition or disorder, for example, refers to reversing the pathology and/or symptoms, such as decreasing the severity of the disease.

The term “prevent,” “preventing” or “prevention” as used herein, comprises the prevention of at least one symptom associated with or caused by the disease, condition or disorder being prevented.

As used herein, “subject” refers to an animal, preferably a mammal, and in particular a human or a non-human animal including livestock animals and domestic animals including, but not limited to, cattle, horses, sheep, swine, goats, rabbits, cats, dogs, and other mammals in need of treatment. In some embodiments, the subject is a human.

As used herein, the term “administration” and variants thereof (e.g., “administering”) in reference to the compound of formula (I) means providing the compound to a subject in need of treatment. As used herein, “orally” and variants thereof (e.g., “oral”) refers to administration via the mouth, i.e., administration of the compound of formula (I) through the mouth.

Administering of the compound of formula (I) to the subject includes both self-administration and administration to the subject by another. The subject may be in need of, or desire, treatment for an existing disease or medical condition, or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

Unless specifically stated or obvious from context, as used herein, the term “about” is understood as within 10%, 9%, 8%, 7%, 6%, 5%, 4%, 3%, 2%, 1%, 0.5%, 0.1%, 0.05%, or 0.01% of the stated value.

In an embodiment, the amount administered to the subject is from about 5 mg to about 300 mg of the compound of formula (I). Whole and half integers between 5 and 300 mg are included in this invention. In an embodiment, the amount administered is from about 10 mg to about 300 mg of the compound of formula (I). In an embodiment, the amount administered is about 10 mg or about 20 mg of the compound of formula (I). In an embodiment, the amount administered is about 5, about 6, about 10, about 12, about 15, about 18, about 20, about 24, about 25, about 30 mg, about 35 mg or about 100 mg of the compound of formula (I). In an embodiment, the amount administered is about 10, about 12, about 15, about 18, about 20, about 24, about 25, or about 30 mg of the compound of formula (I). In an embodiment, the amount administered is about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of the compound of formula (I). In an embodiment, the amount administered is a daily dose of about 5 mg to about 300 mg. In an embodiment, the amount administered is a daily dose of about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of the compound of formula (I). In an embodiment, the amount administered is a daily dose of about 5, about 6, about 10, about 12, about 15, about 18, about 20, about 24, about 25, or about 30 mg of the compound of formula (I). In an embodiment, the amount administered is a daily dose of about about 10, about 12, about 15, about 18, about 20, about 24, about 25, or about 30 mg of the compound of formula (I).

In an embodiment, the amount of formula (I) administered to the subject is from about 10 mg to about 30 mg of the compound of formula (I). In another embodiment, the amount administered to the subject is from about 12 mg to about 27 mg of the compound of formula (I). In still another embodiment, the amount administered to the subject is from about 15 mg to about 25 mg of the compound of formula (I). In an embodiment, the amount administered to the subject is from about 10 mg to about 20 mg of the compound of formula (I). In yet another embodiment, the amount administered to the subject is from about 15 mg to about 20 mg of the compound of formula (I).

In an embodiment, the amount administered to the subject is from about 10 mg to about 30 mg of Compound 1. In another embodiment, the amount administered to the subject is from about 12 mg to about 27 mg of Compound 1. In still another embodiment, the amount administered to the subject is from about 15 mg to about 25 mg of of Compound 1. In an embodiment, the amount administered to the subject is from about 10 mg to about 20 mg of of Compound 1. In yet another embodiment, the amount administered to the subject is from about 15 mg to about 20 mg of Compound 1. In an embodiment, the amount is a daily dose of about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of Compound 1. In an embodiment, the amount is a daily dose of about 15, about 15.5, about 16, about 16.5, about 17, about 17.5, about 18, about 18.5, about 19, about 19.5, about 20, about 20.5, about 21, about 21.5, about 22 mg of Compound 1. In yet another embodiment, the amount administered to the subject in need is about 15 mg, about 17.5 mg, 18 mg, about 20 mg or about 22 mg of Compound 1.

In an embodiment, the amount administered to the subject is from about 10 mg to about 30 mg of Compound 2. In another embodiment, the amount administered to the subject is from about 12 mg to about 27 mg of Compound 2. In still another embodiment, the amount administered to the subject is from about 15 mg to about 25 mg of of Compound 2. In an embodiment, the amount administered to the subject is from about 10 mg to about 20 mg of of Compound 2. In yet another embodiment, the amount administered to the subject is from about 15 mg to about 20 mg of Compound 2. In an embodiment, the amount is a daily dose of about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of Compound 2. In an embodiment, the amount is a daily dose of about 15, about 15.5, about 16, about 16.5, about 17, about 17.5, about 18, about 18.5, about 19, about 19.5, about 20, about 20.5, about 21, about 21.5, about 22 mg of Compound 2. In yet another embodiment, the amount administered to the subject in need is about 15 mg, about 17.5 mg, 18 mg, about 20 mg or about 22 mg of Compound 2.

In an embodiment, the amount administered to the subject is from about 10 mg to about 30 mg of Compound 3. In another embodiment, the amount administered to the subject is from about 12 mg to about 27 mg of Compound 3. In still another embodiment, the amount administered to the subject is from about 15 mg to about 25 mg of of Compound 3. In an embodiment, the amount administered to the subject is from about 10 mg to about 20 mg of of Compound 3. In yet another embodiment, the amount administered to the subject is from about 15 mg to about 20 mg of Compound 3. In an embodiment, the amount is a daily dose of about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 26.5, about 27, about 27.5, about 28, about 28.5, about 29, about 29.5, or about 30 mg of Compound 3. In an embodiment, the amount is a daily dose of about 15, about 15.5, about 16, about 16.5, about 17, about 17.5, about 18, about 18.5, about 19, about 19.5, about 20, about 20.5, about 21, about 21.5, about 22 mg of Compound 3. In yet another embodiment, the amount administered to the subject in need is about 15 mg, about 17.5 mg, 18 mg, about 20 mg or about 22 mg of Compound 3.

In an embodiment, the methods of the instant invention comprise administering an oral dosage form comprising the amount of the compound of formula (I). In a further embodiment, the method comprises administering a single oral dosage form comprising the amount of the compound of formula (I). In further embodiment, the method comprises administering a single oral dosage form comprising the amount of the compound of formula (I) once daily.

In an embodiment, the amount is a therapeutically or prophylactically effective amount of the compound of formula (I). As used herein, “therapeutically effective” or “prophylactically effective” in reference to an amount refers to the amount necessary at the intended dosage to achieve the desired therapeutic and/or prophylactic effect for the period of time desired. The desired effect may be, for example, the alleviation, amelioration, reduction or cessation of at least one symptom associated with the treated condition. For example, when treating hypercholesterolemia, reduction of LDL-C is a desired effect. Amounts may vary, as the ordinarily skilled artisan will appreciate, according to various factors, including but not limited to the disease state, age, sex, and weight of the individual, and the ability of the PCSK9 antagonist to elicit the desired effect in the individual. The response may be documented by in vitro assay, in vivo non-human animal studies, and/or further supported from clinical trials.

In an embodiment, orally administering comprises administering a single oral dosage form comprising the amount of the compound of formula (I). In an embodiment, orally administering comprises administering more than one or multiple oral dosage forms, each comprising the amount of the compound of formula (I) or a portion thereof. In an embodiment, orally administering comprises administering a single oral dosage form comprising the amount of the compound of formula (I) once daily. In an embodiment, orally administering comprises administering more than one or multiple oral dosage forms, each comprising the amount of the compound of formula (I) or a portion thereof, once daily. In an embodiment, orally administering comprises administering a single oral dosage form comprising the amount of the compound of formula (I) more than once daily, e.g., twice, three times or four times daily. In an embodiment, orally administering comprises administering more than one or multiple oral dosage forms, each comprising the amount of the compound of formula (I) or a portion thereof, more than once daily, e.g., twice, three times or four times daily. The oral dosage form may be administered with or without fasting, i.e., with or without food. In an embodiment of the instant invention, the subject in need of treatment fasts approximately 30 minutes before the administration of a compound of formula (I).

In an embodiment, a single oral dosage form is administered once daily for at least 14 days. In an embodiment, a single oral dosage form is administered once daily for 14 days. In an embodiment, a single oral dosage form is administered once daily for as long as the subject is in need of the treatment.

As used herein, an “oral dosage form” refers to a pharmaceutical formulation, comprising the compound of formula (I) and at least one pharmaceutically acceptable excipient, that is suitable for administration through the mouth of the subject. As used herein, the terms “oral dosage form” and “pharmaceutical composition” are intended to encompass both the combination of the specified ingredients in the specified amounts, and any product which results, directly or indirectly, from combination of the specified ingredients in the specified amounts. An oral dosage form may include the entire amount of the compound of formula (I), e.g., about 5 mg to about 300 mg, which may or may not be a daily dose. An oral dosage form may include a portion of a daily dose of the compound of formula (I).

The oral dosage forms according to the disclosure can be solid, semi-solid or liquid. Such oral dosage forms include, but are not limited to, powders, dispersible granules, mini-tablets, and beads (which can be used, for example, for tableting, encapsulation, or direct administration), pills, tablets, lacquered tablets, sugar-coated tablets, hard and soft capsules including gelatin capsules, lozenges, rapidly dissolving tablets, aqueous, alcoholic or oily solutions, gels, syrups, emulsions or suspensions. The oral dosage forms according to the disclosure may comprise additionally one or more coatings which modify release properties, for example, coatings which impart delayed release or formulations which have extended release properties. Also included in the present disclosure are formulations which are intended to be converted, shortly before use, to a suspension or a solution; examples include, but are not limited to, freeze-dried formulations and liquid formulations adsorbed into a solid absorbent medium. In an embodiment, the oral dosage form is a liquid-filled capsule, e.g., a hard gelatin capsule filled with the compound of formula (I) in a combination of Labrasol R and propylene glycol in, e.g., a 2:1 ratio. In an embodiment, the oral dosage form is a liquid-filled capsule, e.g., a hard gelatin capsule filled with the compound of formula (I) in a combination of Labrasol® and propylene glycol in, e.g., a 2:1 ratio, over-encapsulated with an enteric capsule, e.g., an HPMC Vcaps® Enteric capsule (Capsugel®, Lonza). In an embodiment, the oral dosage form is a suspension, e.g., the compound of formula (I) suspended in a combination of OraBlend SF and propylene glycol in, e.g., a 2:1 ratio. In an embodiment, the oral dosage form is a dry-filled enteric coated capsule, e.g., a dry-filled HPMC Vcaps® Enteric capsule (Capsugel®, Lonza). In an embodiment, the oral dosage form is a tablet. In an embodiment, the oral dosage form is a tablet. In a further embodiment, the oral dosage form is tablet which is film-coated.

As will be appreciated by the ordinarily skilled artisan, a pharmaceutically acceptable excipient is any constituent which adapts the composition to a particular route of administration or aids the processing of a composition into a dosage form without itself exerting an active pharmaceutical effect. In general, compositions comprise more than one pharmaceutically acceptable excipient, and the pharmaceutically acceptable excipient(s) is selected based on the form of the oral dosage form. Examples of pharmaceutically acceptable excipients and methods of manufacture of oral dosage forms such as those mentioned above may be found in A. Gennaro (ed.), Remington: The Science and Practice of Pharmacy, 20th Edition, (2000), Lippincott Williams & Wilkins, Baltimore, MD.

Pharmaceutically acceptable excipients suitable for use in the present disclosure include, without limitation, carriers (such as lactose, starch, starch derivatives, talc, stearic acid or its salts for, e.g., pills, tablets, sugar-coated tablets and hard gelatin capsules; such as fats, waxes, semisolid and liquid polyols, natural or hardened oils, etc. for soft capsules; such as water, physiologically acceptable sodium chloride solution, alcohols, glycerol, polyols, sucrose, invert sugar, glucose, mannitol, vegetable oils, etc. for solutions, emulsions or syrups), fillers, disintegrants, binders, lubricants, pressing aids, wetting agents, stabilizers, emulsifiers, absorption enhancers, penetration enhancers, permeation enhancers, dispersants, preservatives, sweeteners, colorants, flavorings, aromatizers, thickeners, diluents, buffer substances, solvents, solubilizers, agents for achieving a depot effect, salts for altering the osmotic pressure, coating agents and/or antioxidants. A particular pharmaceutically acceptable excipient(s), as well as an amount(s) thereof, is selected for use in an oral dosage form so as to provide the desired amount of the compound of formula (I) in an oral dosage form of acceptable volume such that it can provide a therapeutic serum level of the active for an acceptable period of time in the subject to whom the oral dosage form is administered and such that the oral dosage form will retain biological activity during storage within an acceptable temperature range for an acceptable period of time.

In an embodiment, a pharmaceutical composition of the instant invention contains a diluent selected from a polyethylene glycol (of varying molecular weights above 3000), microcrystalline cellulose , mannitol, starch, dicalcium phosphate, calcium carbonate, sodium carbonate, lactose or combinations thereof. In an embodiment, the pharmaceutical composition of the instant invention contains a diluent selected from macrogol (PEG4000), microcrystalline cellulose, mannitol, lactose or combinations thereof. In a further embodiment, the diluent is selected from macrogol (PEG4000), microcrystalline cellulose or lactose. In an embodiment, a pharmaceutical composition of the instant invention contains a disintegrant selected from croscarmellose sodium, crospovidone, or sodium starch glycolate. In a further embodiment, the disintegrant is croscarmellose sodium. In an embodiment, a pharmaceutical composition of the instant invention contains a glidant selected from silicon dioxide, starch, talc, magnesium stearate, or tricalcium phosphate. In a further embodiment, the glidant is selected from silicon dioxide or tricalcium phosphate. In an embodiment, a pharmaceutical composition of the instant invention contains a lubricant selected from magnesium stearate or sodium stearyl fumerate or both. In an embodiment, a pharmaceutical composition of the instant invention contains a solubilizing agent selected from propylene gylcol, polysorbate 80, sorbitol, cremophor EL, castor oil, corn oil, cottonseed oil, safflower oil, sesame oil, soybean oil, peppermint oil, olive oil, miglyol, glycerin or combinations thereof. In an further embodiment, the solubilizing agent is a propylene glycol.

In an embodiment, an oral dosage form further comprises a permeation enhancer. As used herein, a “permeation enhancer” refers to a pharmaceutically acceptable excipient which improves the absorption of an active agent, e.g., the compound of formula (I), from the gastrointestinal tract. Permeation enhancers afford the absorption of cell-impermeable compounds by promoting size-limited passage through tight junctions between intestinal epithelial cells. (D.J. Drucker, Advances in oral peptide therapeutics, Nat Rev Drug Discov, 19, pp 277-289 (2020). Suitable permeation enhancers include, without limitation, sodium caprate, Labrasol®, salcaprozate sodium (SNAC) and combinations thereof. Labrasol® is also known as Caprylocaproyl macrogol-8 glycerides and is manufactured by Gattefosse, Saint Priest, Lyon, France. In an embodiment, an oral dosage form comprises Labrasol®. In an embodiment, an oral dosage form comprises sodium caprate. When present in an oral dosage form, an amount of up to 1800 mg, an amount of up to about 720 mg, an amount of up to about 540 mg, an amount of up to about 360 mg, an amount ranging from about 90 mg to about 360 mg, an amount ranging from about 180 to about 360 mg, or an amount of 90 mg, 180 mg or 360 mg of permeation enhancer is used. In an embodiment of the instant invention, an oral dosage form comprises an amount of up to about 360 mg, an amount ranging from about 90 mg to about 360 mg, an amount ranging from about 180 to about 360 mg, or an amount of 90 mg, 180 mg or 360 mg of a permeation enhancer. In an embodiment, the oral dosage form of the instant invention comprises an amount of 90 mg, 180 mg or 360 mg of a permeation enhancer. In an embodiment, the oral dosage form of the instant invention comprises an amount of 180 mg or 360 mg of a permeation enhancer.

When present in an oral dosage form, an amount of up to about 360 mg, an amount ranging from about 90 mg to about 360 mg, an amount ranging from about 180 to about 360 mg, or an amount of 90 mg, 180 mg or 360 mg of sodium caprate is used. In an embodiment, the oral dosage form of the instant invention comprises the permeation enhancer sodium caprate in the amount of 90 mg, 180 mg or 360 mg. In an embodiment, 180 mg of sodium caprate is used in the oral dosage form. In an embodiment, 360 mg of sodium caprate is used in the oral dosage form.

In an embodiment of the instant invention, dry filled capsules or tablets may be used to administer the compound of formula (I) to a subject in need. In a pharmaceutial composition of the instant invention, a permeation enhancer may be included. In an embodiment, the amount of a permeation enhancer, such as sodium caprate, can range from 1 wt % to 75 wt %. As used herein, wt % refers to the weight percent of an ingredient relative to the total weight of the pharmaceutical composition. In another embodiment, the amount of a permeation enhancer in the pharmaceutical composition is from about 18 wt % to about 65 wt %. For a tablet, the amount of permeation enhancer, such as sodium caprate, may range from about 22 wt % to about 65 wt %. Oral dosage forms may be manufactured by standard methods, includinsonsg wet and dry granulation.

TABLE 1
Examples of formulations using Compound 1

| | | Formulation 1* | Formulation 2* | Formmulation 3** |
|---|---|---|---|---|
| | | Wt % | Wt % | Wt % |
| Formula I | Active | 1-4 | 1 to 7 | 1-2 |
| | Pharmaceutical | | | |
| | Ingredient (API) | | | |
| Sodium Caprate | Permeation | 22-64 | 30 to 75 | 0 |
| | enhancer | | | |
| Labrasol ® | Permeation | 0 | 0 | 60-70 |
| (Caprylocaproyl | enhancer | | | |
| macrogol-8 glycerides) | | | | |
| Macrogol (Polyethylene | Diluent | 0-22 | 0 | 0 |
| glycol (PEG) 4000) | | | | |
| Microcrystalline | Diluent | 0-36 | 20 to 65 | 0 |
| cellulose | | | | |
| Lactose ^{‡} | Diluent | 10-36 | 0 to 45 | 0 |
| Propylene Glycol | Vehicle | 0 | 0 | 30-40 |
| Croscarmellose sodium | Disintegrant | 0-3 | 0 to 5 | 0 |
| Silicon Dioxide | Glidant | 0-2 | 1 to 3 | 0 |
| Magnesium Stearate | Lubricant | 0-2 | 0.5 to 2.0 | 0 |

*Formulations may be compressed into a tablet or filled into a capsule shell not limited to enteric, hard gelatin, or hypromellose
**Filled into hard gelatin capsule

In an embodiment, the instant invention is a pharmaceutical composition comprising a compound of formula (I)

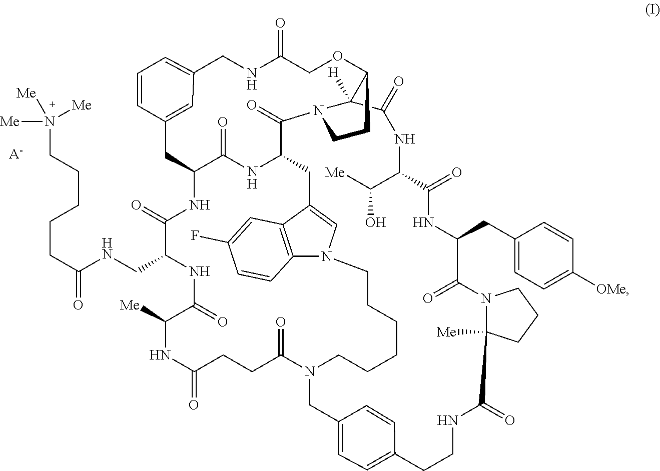

wherein A⁻ is a pharmaceutically acceptable anion, and a permeation enhancer. In a further embodiment, the permeation enhancer is sodium caprate. In another embodiment, the pharmaceutical composition further comprises a diluent. In a futher embodiment, the composition comprises two or more diluents, wherein the two or more diluent comprise a combination of microcrystalline cellulose, macrogol (PEG 4000) and lactose.

In an embodiment of the invention, the pharmaceutical composition comprises a) 1% to 7% by weight relative to the total weight of the pharmaceutical composition of a compound of formula (I); b) about 1% to 75% by weight relative to the total weight of the pharmaceutical composition of a permeation enhancer; c) at least one diluent; d) optionally a glidant and/or a lubricant. In an embodiment, about 18% to 74% by weight relative to the total weight of the pharmaceutical composition of a permeation enhancer is present in the pharmaceutical composition. In another embodiment of the invention, a pharmaceutical composition comprises a) about 1% to 7% by weight relative to the total weight of the pharmaceutical composition of a compound of formula (I); b) about 22% to 67% by weight relative to the total weight of the pharmaceutical composition of a permeation enhancer selected from sodium caprate or Labrasol®; c) at least one diluent or solubilizing agent selected from PEG4000, microcrystalline cellulose, propylene glycol and lactose; d) optionally a glidant; and e) optionally a lubricant.

In an embodiment of the invention, a pharmaceutical composition comprises a) about 2% to 6% by weight relative to the total weight of the pharmaceutical composition of a compound of formula (I); b) about 18% to 74% by weight relative to the total weight of the pharmaceutical composition of a permeation enhancer, where the permeation enhancer is sodium caprate; c) at least one diluent selected from PEG4000, microcrystalline cellulose or lactose; d) 0% to about 3% by weight relative to the total weight of the pharmaceutical composition of a glidant, where the glidant is silicon dioxide; e) 0% to about 2% by weight relative to the total weight of the pharmaceutical composition of a lubricant where the lubricant is magnesium stearate and f) optionally at least one disintegrant.

In an embodiment, the subject has a history of treatment of hypercholesterolemia with one or more statin agents, which has or has not been discontinued; in other words, the subject treated with the compound of formula (I) is currently or was previously treated with statin therapy. In an embodiment, the subject is statin-naïve; in other words, the subject has never been treated with statin therapy. In an embodiment, the subject is concurrently being treated with statin therapy, which has or has not achieved its therapeutic goal.

In an embodiment, one or more additional pharmacologically active agents may be administered in combination with the compound of formula I. As used herein, an “additional pharmacologically active agent(s)” is intended to mean a pharmaceutically active agent(s) that is active in the body, including pro-drugs that convert to pharmaceutically active form after administration, which are different from the compound of formula I, and also includes free-acid, free-base and pharmaceutically acceptable salts of the additional pharmacologically active agents. Generally, any suitable additional pharmacologically active agent(s), including but not limited to anti-hypertensive agents, anti-atherosclerotic agents such as a lipid modifying compound, anti-diabetic agents and/or anti-obesity agents may be used in any combination with the compound of formula I in a single oral dosage form (a fixed dose drug combination) or may be administered to the subject in one or more separate dosage formulations, which allows for concurrent or sequential administration of the compound of formula (I) and the additional pharmacologically active agent(s) (co-administration of the separate active agents).

Examples of additional pharmacologically active agents which may be employed include, but are not limited to, angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan i.e., COZAAR®, valsartan, candesartan, olmesartan, telmesartan and any of these drugs used in combination with hydrochlorothiazide such as HYZAAR®), neutral endopeptidase inhibitors (e.g., thiorphan and phosphoramidon), aldosterone antagonists, aldosterone synthase inhibitors, renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives, peptidyl amino diols and peptidyl beta-aminoacyl aminodiol carbamates, and small molecule renin inhibitors (including diol sulfonamides and sulfinyls), N-morpholino derivatives, N-heterocyclic alcohols and pyrolimidazolones, pepstatin derivatives and fluoro- and chloro-derivatives of statone-containing peptides, enalkrein, RO 42-5892, A 65317, CP 80794, ES 1005, ES 8891, SQ 34017, aliskiren (2(S),4(S),5(S), 7(S)-N-(2-carbamoyl-2-methylpropyl)-5-amino-4-hydroxy-2, 7-diisopropyl-8-[4-methoxy-3-(3-methoxypropoxy)-phenyl]-octanamid hemifumarate) SPP600, SPP630 and SPP635), endothelin receptor antagonists, phosphodiesterase-5 inhibitors (e.g. sildenafil, tadalfil and vardenafil), vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazocin or alpha methyldopa), central alpha adrenergic agonists, peripheral vasodilators (e.g. hydralazine), lipid lowering agents, e.g., HMG-COA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-COA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), fluvastatin (particularly the sodium salt sold in LESCOL®), crivastatin, and pitavastatin, a cholesterol absorption inhibitor such as ezetimibe (ZETIA®) and ezetimibe in combination with any other lipid lowering agents such as the HMG-COA reductase inhibitors noted above and particularly with simvastatin (VYTORIN®) or with atorvastatin calcium, niacin in immediate-release or controlled release forms and/or with an HMG-CoA reductase inhibitor, niacin receptor agonists such as acipimox and acifran, as well as niacin receptor partial agonists, metabolic altering agents including insulin and insulin mimetics (e.g., insulin degludec, insulin glargine, insulin lispro), dipeptidyl peptidase-IV (DPP-4) inhibitors (e.g., sitagliptin, alogliptin, omarigliptin, linagliptin, vildagliptin), insulin sensitizers, including (i) PPARγ agonists, such as the glitazones (e.g. pioglitazone, AMG 131, MBX2044, mitoglitazone, lobeglitazone, IDR-105, rosiglitazone, and balaglitazone), and other PPAR ligands, including (1) PPARα/γ dual agonists (e.g., ZYH2, ZYH1, GFT505, chiglitazar, muraglitazar, aleglitazar, sodelglitazar, and naveglitazar), (2) PPARα agonists such as fenofibric acid derivatives (e.g., gemfibrozil, clofibrate, ciprofibrate, fenofibrate, bezafibrate), (3) selective PPARγ modulators (SPPARγM's), (e.g., such as those disclosed in WO 02/060388, WO 02/08188, WO 2004/019869, WO 2004/020409, WO 2004/020408, and WO 2004/066963); and (4) PPARγ partial agonists, (ii) biguanides, such as metformin and its pharmaceutically acceptable salts, in particular, metformin hydrochloride, and extended-release formulations thereof, such as Glumetza™, Fortamet™M, and GlucophageXR™, and (iii) protein tyrosine phosphatase-1B (PTP-1B) inhibitors (e.g., ISIS-113715 and TTP814), insulin or insulin analogs (e.g., insulin detemir, insulin glulisine, insulin degludec, insulin glargine, insulin lispro and inhalable formulations of each), leptin and leptin derivatives and agonists, amylin and amylin analogs (e.g., pramlintide), sulfonylurea and non-sulfonylurea insulin secretagogues (e.g., tolbutamide, glyburide, glipizide, glimepiride, mitiglinide, meglitinides, nateglinide and repaglinide), α-glucosidase inhibitors (e.g., acarbose, voglibose and miglitol), glucagon receptor antagonists (e.g., MK-3577, MK-0893, LY-2409021 and KT6-971), incretin mimetics, such as GLP-1, GLP-1 analogs, derivatives, and mimetics, GLP-1 receptor agonists (e.g., dulaglutide, semaglutide, albiglutide, exenatide, liraglutide, lixisenatide, taspoglutide, CJC-1131, and BIM-51077, including intranasal, transdermal, and once-weekly formulations thereof), bile acid sequestering agents (e.g., colestilan, colestimide, colesevalam hydrochloride, colestipol, cholestyramine, and dialkylaminoalkyl derivatives of a cross-linked dextran), acyl CoA:cholesterol acyltransferase inhibitors (e.g., avasimibe), antiobesity compounds, agents intended for use in inflammatory conditions, such as aspirin, non-steroidal anti-inflammatory drugs or NSAIDs, glucocorticoids, and selective cyclooxygenase-2 or COX-2 inhibitors, glucokinase activators (GKAs) (e.g., AZD6370), inhibitors of 11β-hydroxysteroid dehydrogenase type 1 (e.g., such as those disclosed in U.S. Pat. No. 6,730,690, and LY-2523199), CETP inhibitors (e.g., anacetrapib, torcetrapib, and evacetrapib), inhibitors of fructose 1,6-bisphosphatase (e.g., such as those disclosed in U.S. Pat. Nos. 6,054,587; 6,110,903; 6,284,748; 6,399,782; and 6,489,476), inhibitors of acetyl CoA carboxylase-1 or 2 (ACC1 or ACC2), AMP-activated Protein Kinase (AMPK) activators, other agonists of the G-protein-coupled receptors: (i) GPR-109, (ii) GPR-119 (e.g., MBX2982 and PSN821), and (iii) GPR-40 (e.g., TAK875), SSTR3 antagonists (e.g., such as those disclosed in WO 2009/001836), neuromedin U receptor agonists (e.g., such as those disclosed in WO 2009/042053, including, but not limited to, neuromedin S (NMS)), SCD modulators, GPR-105 antagonists (e.g., such as those disclosed in WO 2009/000087), SGLT inhibitors (e.g., ASP1941, SGLT-3, empagliflozin, dapagliflozin, canagliflozin, BI-10773, ertugliflozin, remogloflozin, TS-071, tofogliflozin, ipragliflozin, and LX-4211), inhibitors of acyl coenzyme A:diacylglycerol acyltransferase 1 and 2 (DGAT-1 and DGAT-2), inhibitors of fatty acid synthase, inhibitors of acyl coenzyme A:monoacylglycerol acyltransferase 1 and 2 (MGAT-1 and MGAT-2), agonists of the TGR5 receptor (also known as GPBARI, BG37, GPCR19, GPR131, and M-BAR), ileal bile acid transporter inhibitors, PACAP, PACAP mimetics, and PACAP receptor 3 agonists, PPAR agonists, protein tyrosine phosphatase-1B (PTP-1B) inhibitors, IL-1b antibodies (e.g., XOMA052 and canakinumab), bromocriptine mesylate and rapid-release formulations thereof, and bempedoic acid, as well as other drugs beneficial for the treatment of the above-mentioned conditions or disorders including the free-acid, free-base, and pharmaceutically acceptable salt forms of the above additional pharmacologically active agents where chemically possible.

In an embodiment, the additional pharmacologically active agent is a statin, ezetimibe, bempedoic acid, any other cholesterol lowering agent considered to be standard of care, or any combination thereof.

In an embodiment, the methods of the disclosure further comprise the step of administering a statin agent. Hence, the compound of formula (I) will be co-administered with at least one statin agent. The compound of formula (I) may be administered with the statin agent simultaneously or separately. This administration in combination can include simultaneous administration of the compound of formula (I) and the statin agent in the same oral dosage form, simultaneous administration in separate dosage forms, and separate administration. That is, the compound of formula (I) and the statin agent can be formulated together in the same oral dosage form and administered simultaneously. Alternatively, the compound of formula (I) and the statin agent can be simultaneously administered, wherein both are present in separate formulations. In another alternative, the compound of formula (I) can be administered just followed by the statin agent, or vice versa. In some embodiments of the separate administration protocol, the compound of formula (I) and the statin agent are administered a few minutes apart, or a few hours apart, or a few days apart.

In an embodiment, a level of LDL-C of the subject after treating with the compound of formula (I) is reduced from a baseline level of LDL-cholesterol before treating with the compound of formula (I). In an embodiment, the level of LDL-C of the subject after treating with the compound of formula (I) is reduced by at least 10%, at least 20%, at least 30%, at least 40%, at least 50%, more than 50%, at least 60%, more than 60%, at least 65%, more than 65%, at least 70%, more than 70%, at least 75%, more than 75%, at least 80%, more than 80%, at least 85%, more than 85%, or at least 90% from the baseline level of LDL-C before treating with the compound of formula (I). In an embodiment, the level of LDL-C of the subject after treating with the compound of formula (I) is reduced by more than 50% from the baseline level of LDL-C before treating with the compound of formula (I). In an embodiment, the level of LDL-C of the subject after treating with the compound of formula (I) is reduced by more than 60% from the baseline level of LDL-C before treating with the compound of formula (I). In an embodiment, the level of LDL-C of the subject after treating with the compound of formula (I) is reduced by more than 65% from the baseline level of LDL-C before treating with the compound of formula (I). In an embodiment, the level of LDL-C of the subject after treating with the compound of formula (I) is reduced by more than 70% from the baseline level of LDL-C before treating with the compound of formula (I). In an embodiment, the level of LDL-C of the subject was reduced by more than 50% from the baseline level of LDL-C after 14 days of treating the subject with the compound of formula (I). In an embodiment, the level of LDL-C of the subject was reduced by more than 60% from the baseline level of LDL-C after 14 days of treating the subject with the compound of formula (I). In an embodiment, the level of LDL-C of the subject was reduced by more than 65% from the baseline level of LDL-C after 14 days of treating the subject with the compound of formula (I). In an embodiment, the level of LDL-C of the subject was reduced by more than 70% from the baseline level of LDL-C after 14 days of treating the subject with the compound of formula (I). Both the baseline level and after-treatment level of LDL-C may be determined by standard clinical laboratory tests used to measure blood cholesterol.

In an embodiment, the level of LDL-C of the subject after treating with the compound of formula (I) is reduced by at least 50% from the baseline level of LDL-C before treating with the compound of formula (I). In an embodiment, the level of LDL-C of the subject after treating with the compound of formula (I) is reduced by at least 60% from the baseline level of LDL-C before treating with the compound of formula (I). In an embodiment, the level of LDL-C of the subject after treating with the compound of formula (I) is reduced by at least 65% from the baseline level of LDL-C before treating with the compound of formula (I). In an embodiment, the level of LDL-C of the subject after treating with the compound of formula (I) is reduced by at least 70% from the baseline level of LDL-C before treating with the compound of formula (I). In an embodiment, the level of LDL-C of the subject was reduced by at least 50% from the baseline level of LDL-C after 14 days of treating the subject with the compound of formula (I). In an embodiment, the level of LDL-C of the subject was reduced by at least 60% from the baseline level of LDL-C after 14 days of treating the subject with the compound of formula (I). In an embodiment, the level of LDL-C of the subject was reduced by at least 65% from the baseline level of LDL-C after 14 days of treating the subject with the compound of formula (I). In an embodiment, the level of LDL-C of the subject was reduced by at least 70% from the baseline level of LDL-C after 14 days of treating the subject with the compound of formula (I). Both the baseline level and after-treatment level of LDL-C may be determined by standard clinical laboratory tests used to measure blood cholesterol.

In an embodiment, the invention is directed to a method of lowering the Apolipoprotein B (Apo B) level of a subject in need thereof comprising orally administering to the subject an amount of a compound of formula (I). In some embodiments, after treating with the compound of formula (I), the Apolipoprotein B (Apo B) level of a subject in need thereof is reduced from a baseline level of Apo B before treating with the compound of formula (I). In an embodiment, the level of Apo B of the subject after treating with the compound of formula (I) is reduced by at least 10%, at least 20%, at least 30%, at least 40%, at least 50%, more than 50%, at least 60%, more than 60%, at least 65%, more than 65%, at least 70%, more than 70%, at least 75%, more than 75%, at least 80%, more than 80%, at least 85%, more than 85%, or at least 90% from the baseline level of Apo B before treating with the compound of formula (I).

In an embodiment, the level of non-high density lipoprotein cholesterol (non-HDL-C) of the subject after treating with the compound of formula (I) is reduced from a baseline level of non-HDL-C before treating with the compound of formula (I). In an embodiment, the level of non-HDL-C of the subject after treating with the compound of formula (I) is reduced by at least 10%, at least 20%, at least 30%, at least 40%, at least 50%, more than 50%, at least 60%, more than 60%, at least 65%, more than 65%, at least 70%, more than 70%, at least 75%, more than 75%, at least 80%, more than 80%, at least 85%, more than 85%, or at least 90% from the baseline level of non-HDL-C before treating with the compound of formula (I).

Inhibition or antagonism of one or more of PCSK9-associated functional properties can be readily determined according to methodologies known to the art (see, e.g., Barak & Webb, 1981 J. Cell Biol. 90:595-604; Stephan & Yurachek, 1993 J. Lipid Res. 34:325330; and McNamara et al., 2006 Clinica Chimica Acta 369:158-167) as well as those described herein. Inhibition or antagonism will effectuate a decrease in PCSK9 activity relative to that seen in the absence of the antagonist or, for example, that seen relative to the activity observed when a control antagonist of irrelevant specificity is present. Preferably, the compound of formula (I) antagonizes PCSK9 functioning to the point that there is a decrease of at least 10%, of the measured parameter including but not limited to the activities disclosed herein, and more preferably, a decrease of at least 20%, 30%, 40%, 50%, 60%, 65%, 70%, 75%, 80%, 85%, 90% and 95% of the measured parameter.

The compound of formula (I) has been shown to be highly effective at lowering LDL cholesterol and generally well tolerated following single and multiple oral doses in healthy volunteers. The compound of formula (I) reduced levels of free PCSK9 protein, which contributes to high LDL cholesterol, by more than 90% from baseline following treatment with single doses of the compound of formula (I). Following 14 days of once daily oral dosing, the compound of formula (I) lowered LDL-cholesterol in the blood by approximately 65% from baseline levels in participants already on a background of moderate-to-high intensity statin therapy. These participants were already taking statin medications to control their cholesterol levels. The compound of formula (I) may be a highly effective treatment for patients suffering from high cholesterol.

These examples are provided for the purpose of further illustration only and are not intended to be limitations on the disclosure.

### EXAMPLES

### Example 1

### Preparation of Compound 1 (Amorphous Chloride Salt)

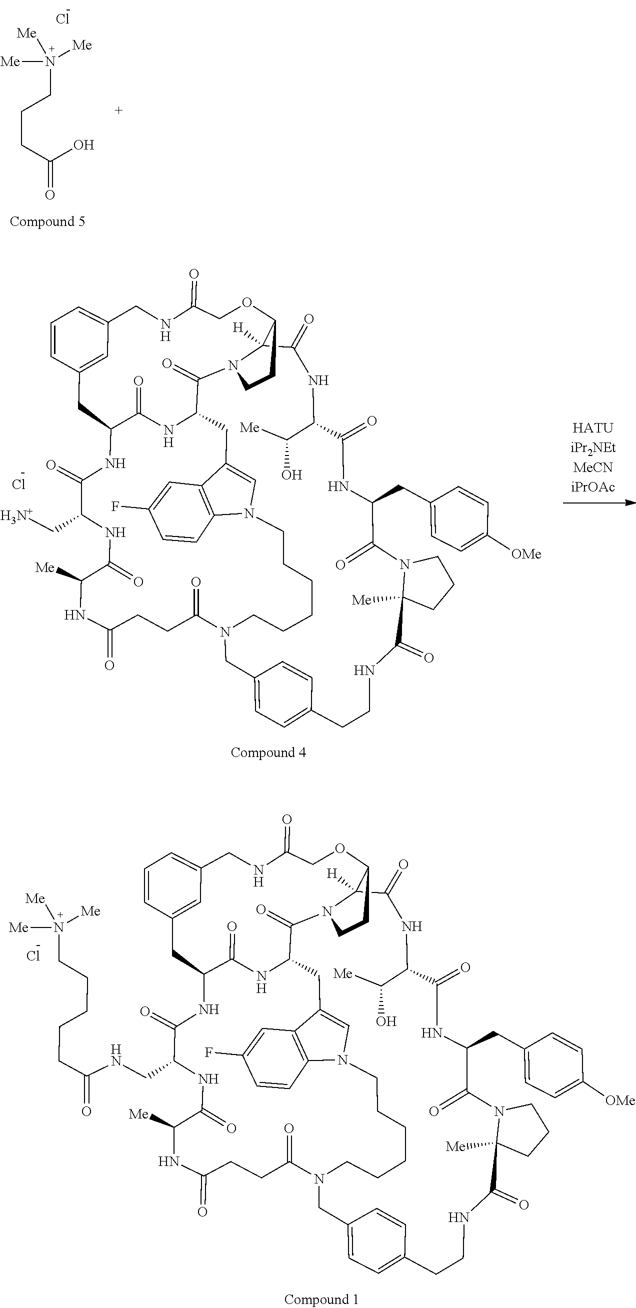

A 50 L cylindrical reactor was charged with 0.5 L of acetonitrile (MeCN), followed by compound 4 (294.7 g, 206 mmol) at room temperature. (Methods of synthesizing the starting material Compound 4 are described in WO2019/246349, see Ex-01.) Additional 2.4 L of MeCN was used to rinse all the solids to the bottom of the reactor. Compound 5 (5-carboxy-N,N,N-trimethylpentan-1-aminium chloride, 47.5 g, 227 mmol) was added. Additional 2.0 L of MeCN was used to rinse all the solids to the bottom of the reactor. N,N-diisopropylethylamine (iPr₂NEt, 216 mL, 1236 mmol) was added and 0.5 L of MeCN was used to rinse the liquid to the bottom of the reactor. The reactor was charged with 1-[bis(dimethylamino)methylene]-1H-1,2,3-triazolo[4,5-b]pyridinium-3-oxide hexafluorophosphate (HATU, 94 g, 247 mmol) and 0.5 L of MeCN was used to rinse all the solids to the bottom of the reactor. After 3 h at room temperature, isopropyl acetate (iPrOAc, 17.7 L) was added dropwise over 1 h. The slurry was filtered and the wet cake was washed three times with 2.9 L of iPrOAc. The solids were dried under vacuum with a N₂ sweep to provide 337 g of the crude product.

The crude product was purified using supercritical fluid chromatography (stationary phase: DIACEL DCpak P4VP [30×250 mm, 5 μm]; mobile phase: 45% modifier (0.25% NH₄OH and 5% H₂O in MeOH) and 55% CO₂). Fractions containing the product were concentrated using rotary evaporation. The residue after evaporation was dissolved in water (3.2 L) and 0.1 M aqueous HCl (1389 ml, 139 mmol) was added at room temperature (pH at the end of addition was measured as 6 using pH paper). The resulting solution was filtered through 0.22 μm line filter and the filtrate was lyophilized to provide 238 g of Compound 1 (amorphous chloride salt).

### Example 2

### Preparation of Lyophilized Compound 2 (Amorphous Caprate Salt)

Macroporous anion exchange resin AG MP-1M (6 g, 100-200 mesh, chloride form) was packed in a 60 mL funnel. The packed resin was washed with 9 mL of a mixture of acetonitrile and water (1:1), five times. The resin was washed with 200 mL of 1 M NaOH and then with 10 mL of water, two times. The resin was transferred to a glass column and washed with 10 mL of water, three times. The resin was then washed with 10 mL of EtOH, two times, and then with 9 mL of IM capric acid solution in EtOH, five times, followed by 9 mL of EtOH, three times. Compound 1 (0.3 g) was dissolved in 6 mL MeCN/water (1:1) and loaded into the resin-packed column. The filtrate was collected in a 20 mL vial. The column was washed with 15 mL of MeCN and water solution (1:1), three times, and the filtrate was collected in 20 ml vials. The fractions containing Compound 2 caprate were combined and concentrated, to remove MeCN, and then the desired amorphous Compound 2 (0.29 g) was isolated via lyophilization.

### Example 3

### Preparation of Lyophilized Compound 3 (Amorphous Acetate Salt)

Macroporous anion exchange resin AG MP-IM (6 g, 100-200 mesh, chloride form) was packed in a 60 mL funnel. The packed resin was washed with 9 mL of the mixture of acetonitrile/water (1:1 ratio), 5 times. The resin was washed with 200 mL of IM NaOH and then with 50 mL of IM AcOH in water. The resin was transferred to a 100 mL round bottom flask containing a solution of Compound 1 (chloride salt, 0.3 g) in 6 mL of a 1:1 mixture of acetonitrile and water. An additional 18 mL of MeCN/water (1:1) was added. The mixture was aged at room temperature for 30 minutes and the resulting mixture was transferred into a 60 mL funnel. The filtrate was collected and the resin was washed with 10 mL MeCN/water (1/1), three times, and the filtrate was collected in 20 ml vials. The fractions containing Compound 3 were combined and concentrated, to remove MeCN, and then the desired amorphous Compound 3 (0.304 g) was isolated via lyophilization of the solution.

### Example 4

### Preparation of Tablets Containing a Compound of Formula (I)

Sodium Caprate (1.5 kg) and Macrogol (499.9 g) were loaded into a 10 L high shear granulator. The two components were dry mixed in the high shear grator for 1 min at an impeller speed of 183 rpm. During continuous mixing in the high shear granulator, water was added until the appropriate degree of granulation was reached. Wet granules were milled through cone mill with a screen size of 2.0 mm, then transferred to a fluid bed dryer and dried using an inlet temperature of 70° C. until the predetermined loss on drying of the granules was reached (<3.00%). Dried granules were milled through a cone mill with a screen size of 1.0 mm. The dried granules (1.275 kg) were then mixed with Compound 1 (26.96 g), lactose (150.4 g), and silicon dioxide (22.58 g) in a 10 L diffusion blender for 920 revolutions, then milled through a cone mill with screen size of 0.8 mm. The milled blend was then mixed with magnesium stearate (22.58 g) in a 10 L diffusion blender for 460 revolutions. The final lubricated granules were compacted into tablets using a rotary press with target weight of 564.9mg.

### Example 5

### Dry Filled Capsule Manufacturing Process

Microcrystalline cellulose (179.7 g), sodium caprate (661 g), Compound 1 (41 g) and silicon dioxide (8.996 g), were mixed using a 10L diffusion blender for 375 revolutions, then magnesium stearate (4.498 g) was added to the blender and mixed for 250 revolutions. The blend was then granulated by roller compaction utilizing 21 bar of roll pressure, a 2.0 mm coarse screen and a 1 mm fine screen. Roller compacted granules (761.9 g) were mixed with magnesium stearate (3.8 g) using a 5 L diffusion blender for 250 revolutions. Final lubricated granules were then manually encapsulated to target fill weight of 490 mg.

### Example 6

### Liquid Filled Capsule Manufacture Process

A solution of Labrasol® ALF (caprylocaproyl macrogol-8 glycerides, 100 mL) and propylene glycol (50 mL) was prepared in a 250 ml bottle using a stir plate as the vehicle. Compound 1 (0.7747 g) was dissolved in the vehicle (49.7 mL) in a 125 ml bottle using a stir plate for 5 min, followed by sonication for 15 min. The final solution was filled into hard gelatin capsules to a target weight of 548 mg. The hard gelatin capsules were then manually sealed using an 50% ethanol in water solution and inspected for leaks. The final hard gelatin capsules were over-encapsulated into enteric capsules which were then manually sealed using an 90% ethanol in water solution. The 50% ethanol in water solution was prepared by mixing 10.4 mL of ethanol (96%) and 9.6 mL of water in a 30 ml bottle using a stir plate for 15 min. The 90% ethanol in water solution was prepared by mixing 18.8 mL of ethanol (96%) and 1.2 mL of water in a 30 mL bottle using a stir plate for 15 min.

### Testing

Chemically stable and resistant to gastrointestinal (GI) degradation, Compound 1, the amorphouse chloride salt of a compound of formula (I),

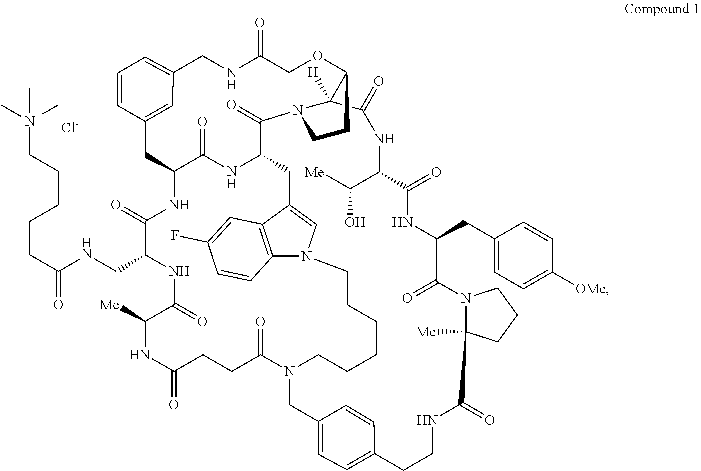

displayed picomolar binding affinity against human PCSK9. GI absorption of Compound 1 was improved with co-administration with a permeation enhancer (Labrasol, sodium caprate) in rats, non-human primates. Preclinical Good Laboratory Practices (GLP) toxicity studies in rats and nonhuman primates support clinical development; these studies were performed using both subcutaneous dosing (to achieve high systemic exposure of Compound 1) plus oral arms (to evaluate local/GI tolerability). In these GLP toxicity studies, no adverse effects were observed up to/including highest doses administered.

### Safety Testing

Evaluation of pharmacokinetics, pharmacodynamics (reduction of free PCSK9 from baseline) and safety and tolerability of single doses of the compound of formula (I) were studied in normal healthy male volunteers aged 18-50. The objectives of the study were to evaluate the safety and tolerability of single doses of Compound 1 (about 10 mg to about 300 mg) and the pharmacokinetics (PK) of Compound 1. Additionally, this trial examined the effect of permeation enhancer dose on PK, the effect of food on PK, and the effect of various capsule formulations on PK. The pharmacodynamic endpoint measured in this study was target engagement (% change in free PCSK9). For each panel in the trial, participants were randomized to receive either Compound 1 or placebo (PBO) in a 9:3 randomization scheme (n=9 Compound 1 : n=3 PBO). Baseline characteristics of the participants in this study are shown in Table 2.

TABLE 2
baseline characteristics of Safety Testing
Baseline Characteristics of Participants

| Gender | |
|---|---|
| Male (n) | 60 |
| Age (years) | |
| Adults (18-50) (n) | 60 |
| Mean | 38.1 |
| SD | 9.2 |
| Median | 40 |
| Range | 19 to 50 |
| Race (n) | |
| American Indian or Alaska Native | 1 |
| Black or African American | 1 |
| White | 58 |
| Ethnicity (n) | |
| Hispanic or Latino | 1 |
| Not Hispanic or Latino | 57 |
| Unknown | 2 |

Single doses of Compound 1, which is the amorphous chloride salt of a compound of formula (I), were administered in liquid-filled hard gelatin capsules. Capsules contained Compound 1 at various strengths, or no Compound 1 (placebo), and a mixture of the liquid permeation enhancer Labrasol® and propylene glycol in a 2:1 ratio, with various amounts up to 1800 mg of Labrasol® being included. The capsules were over-encapsulated with enteric capsules (HPMC Vcaps® Enteric, Capsugel®, Lonza).

This study also evaluated a 40 mg/mL suspension of Compound 1 in OraBlend SF and propylene glycol in a 2:1 ratio with no permeation enhancer, administered via syringe/PO dosing, as well as dry-filled enteric coated capsules (HPMC Vcaps®Enteric, Capsugel®, Lonza) containing various strengths of Compound 1 and sodium caprate up to 1800 mg. The minimum dose of Compound 1 in this trial was 10 mg, and the highest dose administered was 300 mg. Compound 1 was well tolerated at doses up to 300 mg with no deaths, serious adverse events, or clinically meaningful trends in laboratory safety tests, vital signs, or ECGs as a function of study intervention. In this study, there were no death or severe adverse events (SAEs). Out of 60 total participants, there were 6 discontinuations—three due to an adverse event (maculopapular rash, wound associated with concussion/injury, lower back pain), two were due to protocol violations and one was a withdrawal due to a participant's work conflict. Adverse Events (AEs) reported by the Investigator of the study that were related to Compound 1 included abdominal discomfort, diarrhea, dyspepsia, headache, and maculopapular rash. All treatment-related AEs were mild/moderate, with the exception of one participant having severe back pain, which was not dose-related.

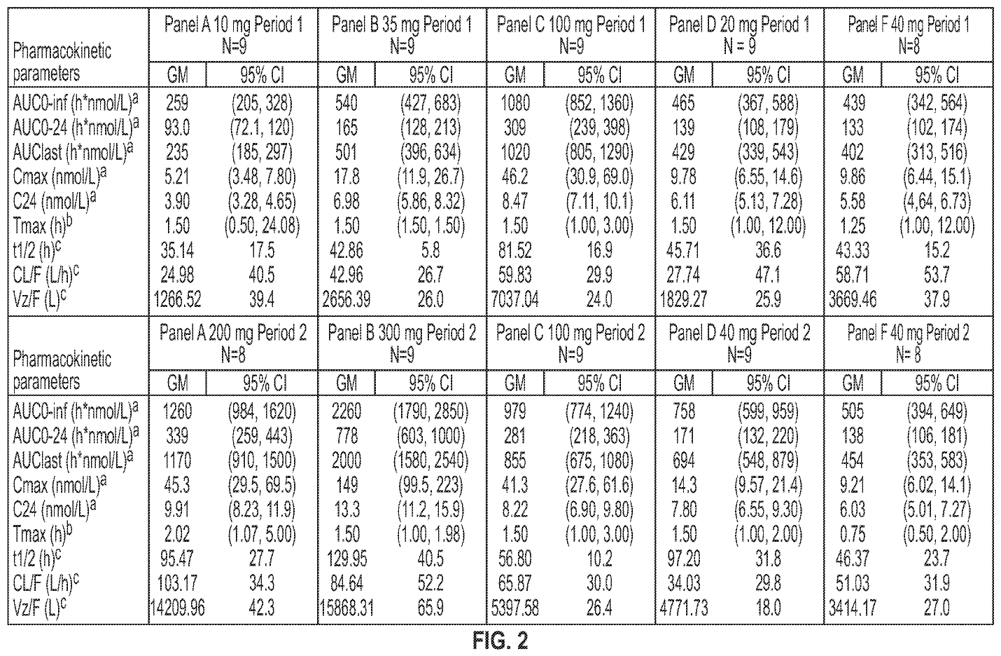

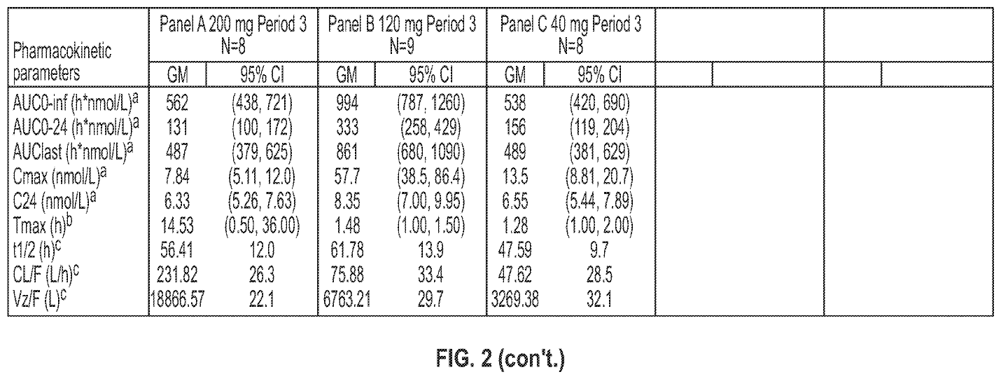

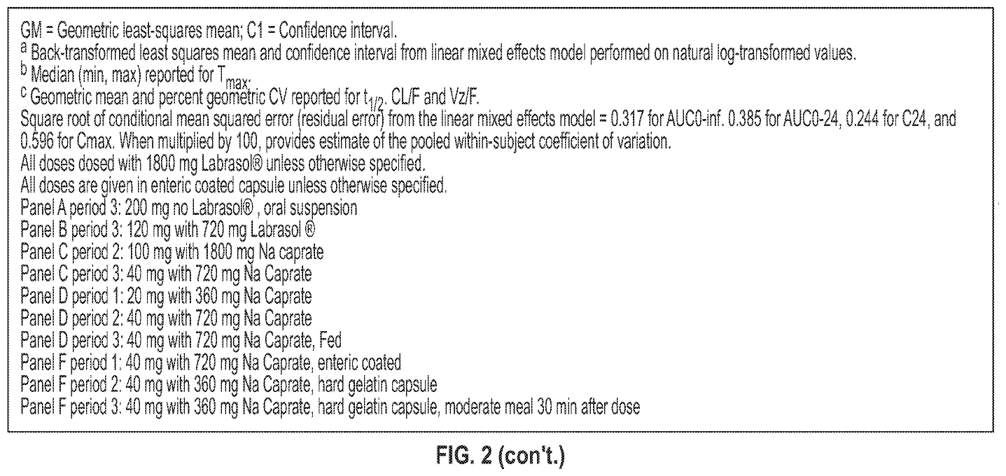

Compound 1 (the amorphous chloride salt of a compound of formula (I)) exhibited a dose dependent increase in plasma exposure and >90% mean maximum reduction of free plasma PCSK9 levels from baseline at all dose levels studied. See FIG. 2 and Table 3 below.

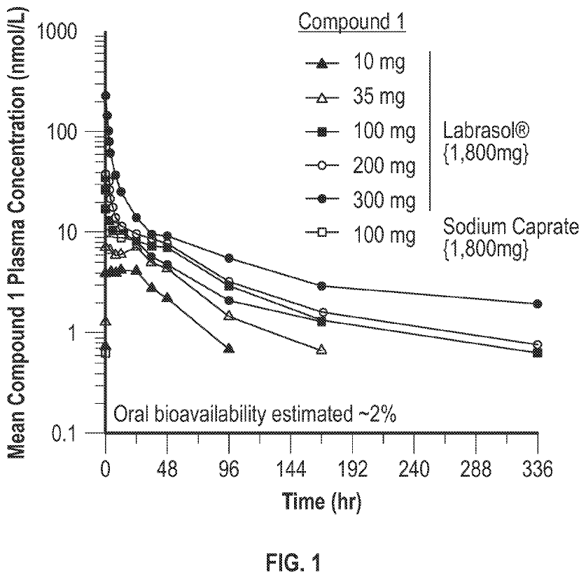

Pharmacokinetic results are shown in FIG. 1. This trial also demonstrated that permeation enhancers improve absorption, evidenced by an increase in the Cmax and AUC0-24 (see FIG. 2). The PK of Compound 1 in the presence of permeation enhancers Labrasol® and sodium caprate was similar (as seen in FIG. 2). This trial also demonstrated that food consumed 30 minutes before dosing resulted in a lower plasma exposure compared to fasted conditions, while food consumed 30 minutes after the dose had a negligible effect on plasma exposure (see FIG. 2).

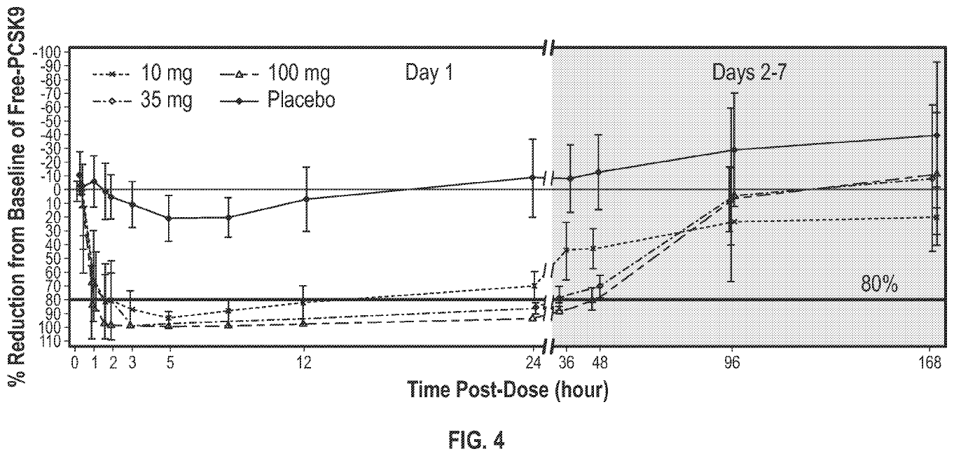

As seen in FIG. 4, dosing of Compound 1 was associated with a reduction in plasma levels of free PCSK9 protein, which contributes to high LDL cholesterol, of greater than 90% compared to baseline levels.

TABLE 3
Model-based Geometric Mean (GM) and 95% Confidence Interval
(CI) for Maximum Percent Target Reduction From Baseline
of Free PCSK9 by Dose With Posterior Probability of True
GM Free PCSK9 Maximum Percent Reduction ≥80%

| Compound 1 Dose | | | | Posterior |
|---|---|---|---|---|
| (mg) | N | GM^{a} | 95% CI^{a} | Probability^{b} |
| Placebo^{c} | 17 | 36.06 | 33.36, 38.99 | 0 |
| Panel A 10 mg | 9 | 93.32 | 84.11, 103.54 | >0.99 |
| Panel B 35 mg | 9 | 98.72 | 88.95, 109.56 | >0.99 |
| Panel C 100 mg | 9 | 99.36 | 89.66, 110.11 | >0.99 |
| Panel A 200 mg | 8 | 99.46 | 89.09, 111.03 | >0.99 |
| Panel B 300 mg | 9 | 99.68 | 89.82, 110.61 | >0.99 |
| Panel C 100 mg with | 9 | 99.18 | 89.50, 109.90 | >0.99 |
| 1800 mg Na caprate | | | | |
| Panel A 200 mg no PE | 8 | 96.83 | 86.74, 108.10 | >0.99 |
| Panel B 120 mg with | 9 | 99.43 | 89.59, 110.34 | >0.99 |
| 720 mg Labrasol ® | | | | |
| Panel C 40 mg with | 8 | 97.94 | 87.80, 109.26 | >0.99 |
| 720 mg Na Caprate | | | | |

CI = confidence interval; GM = geometric mean; PE = permeation enhancer
^{a}Back-transformed least-squares mean and confidence interval from mixed effects model performed on natural log-transformed values.
^{b}Posterior probability that the true GM PCSK9 percent reduction ≥80% of Compound 1 in plasma
^{c}Placebo is pooled over panels across periods.

### LDL-Cholesterol Reduction Testing

Dosing of Compound 1 to achieve a target LDL-C reduction of >50% was assessed using a multiple dose study in male and female participants aged 18-65 receiving statin background therapy to control blood cholesterol. The baseline mean LDL-C for participants was ˜87 mg/dL, and 85% of participants were receiving moderate or high-intensity statins. Either a placebo or Compound 1 was administered once daily for 14 days in the morning after an overnight fast. Standard takeaway meals were provided to participants to consume no less than 30 minutes after receiving their once daily dose. In addition to standard safety monitoring, including vital signs, ECG and laboratory safety tests, plasma lipids (total cholesterol, LDL-C, HDL-C and TG) were measured. LDL-C was monitored as part of the safety labs.

The starting dose of 20 mg of Compound 1 plus 360 mg sodium caprate was associated with a mean reduction of plasma LDL-C of approximately 62%. 10 mg of Compound 1 plus 360 mg sodium caprate was the next dose studied. 10 mg of Compound 1 plus 360 mg sodium caprate was associated with a mean reduction of LDL-C of approximately 64%. The third dose level was also 10 mg of Compound 1; however, the formulation contained 180 mg sodium caprate. The 10 mg of Compound 1 plus 180 mg sodium caprate dose was associated with a mean reduction of LDL-C of approximately 60%. The PK from this dose was similar to that of the 10 mg of Compound 1 plus 360 mg sodium caprate dose, which supported the similarity of LDL-C reduction. All formulations containing either sodium caprate alone (placebo) or both sodium caprate and Compound 1 were in the form of dry-filled enteric coated capsules (HPMC Vcaps® Enteric, Capsugel®, Lonza).

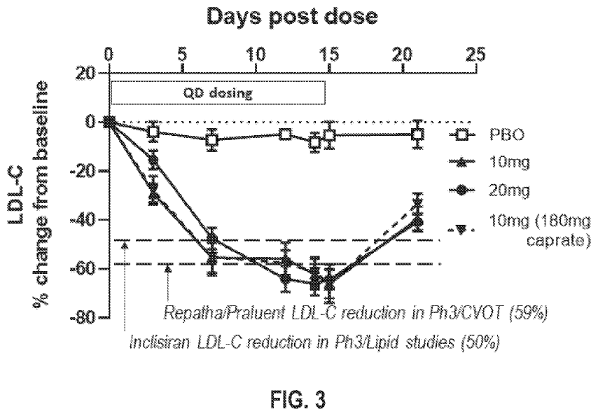

Blood LDL-cholesterol levels were measured using standard clinical laboratory testing procedures predose and on day 3, 7, 14, 15 and 21 after dosing. Results of this study are presented in FIG. 3. As shown therein, the maximum reduction in LDL-cholesterol observed at the 10 and 20 mg doses is in the range of LDL-C reduction observed with anti-PCSK9 monoclonal antibodies Repatha and Praluent, reported in their Phase 3 cardiovascular outcomes trials, and in the Phase 3 lipid trial of the anti-PCSK9 siRNA Inclisiran. See, Repatha cardiovascular outcomes trial FOURIER reporting 59% reduction in LDL-C, N Engl J Med 2017 May 4, 376 (18): 1713-1722; Praluent cardiovascular outcomes trial ODYSSEY reporting 59% reduction in LDL-C, N Engl J Med 2018, 379:2097-2107; and Inclisiran phase 3 lipid trials reporting 49-52% reduction in LDL-C, N Engl J Med 2020, 382:1507-1519. By contrast, placebo-treated participants exhibited <5% LDL-C reduction from baseline.

A hard gelatin capsule containing 5 mg of Compound 1, plus 180 mg sodium caprate, was administered to male and female participants taking statins to control cholesterol in a separate study. The % LDL-C reduction was less than observed in the trial reported above (<50% reduction from baseline).

1. A method of treating hypercholesterolemia in a subject in need of such treatment, comprising orally administering to the subject an amount of a compound of formula (I) 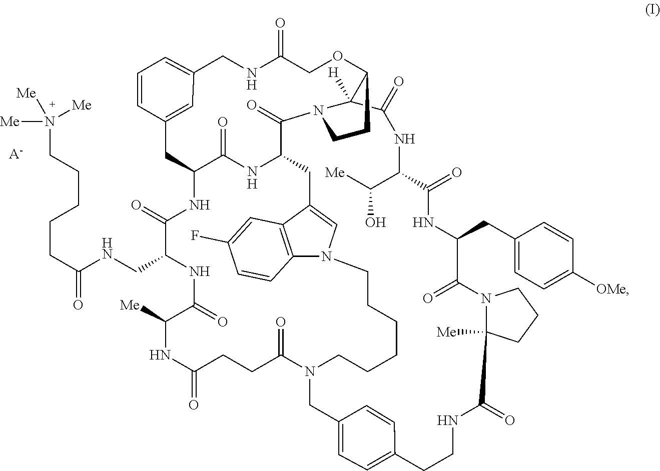
wherein A⁻ is selected from a pharmaceutically acceptable anion, and wherein the amount administered is from about 5 mg to about 300 mg of the compound of formula (I).

2. The method according to claim 1, wherein the amount administered is from about 10 mg to about 30 mg of the compound of formula (I).

3. The method according to claim 1, wherein the amount administered is 10 mg, 12.5 mg, 15 mg, 17.5 mg, 18 mg or 20 mg of the compound of formula (I).

4. The method according to claim 1, wherein the amount administered is 10 mg or 20 mg of the compound of formula (I).

5. The method according to any of claim 1, wherein a compound of formula (I) is administered in an oral dosage form which further comprises a permeation enhancer.

6. The method according to claim 5, wherein the permeation enhancer is sodium caprate.

7. The method according to claim 5, wherein the oral dosage form comprises 180 mg of the permeation enhancer.

8. The method according to any of claim 1, wherein a compound of formula (I) is administered in a single oral dosage form which is administered once daily for at least 14 days.

9. The method according to any of claim 1, wherein the subject is currently or was previously treated with statin therapy.

10. The method according to claim 1, wherein a level of LDL-cholesterol of the subject after treating is reduced from a baseline level of LDL-cholesterol before treating.

11. The method according to claim 10, wherein the level of LDL-cholesterol of the subject after treating is reduced by more than 50% from the baseline level of LDL-cholesterol before treating.

12. The method according to claim 1, wherein the subject is a human.

13. The method according to claim 1, wherein the compound of formula (I) is selected from

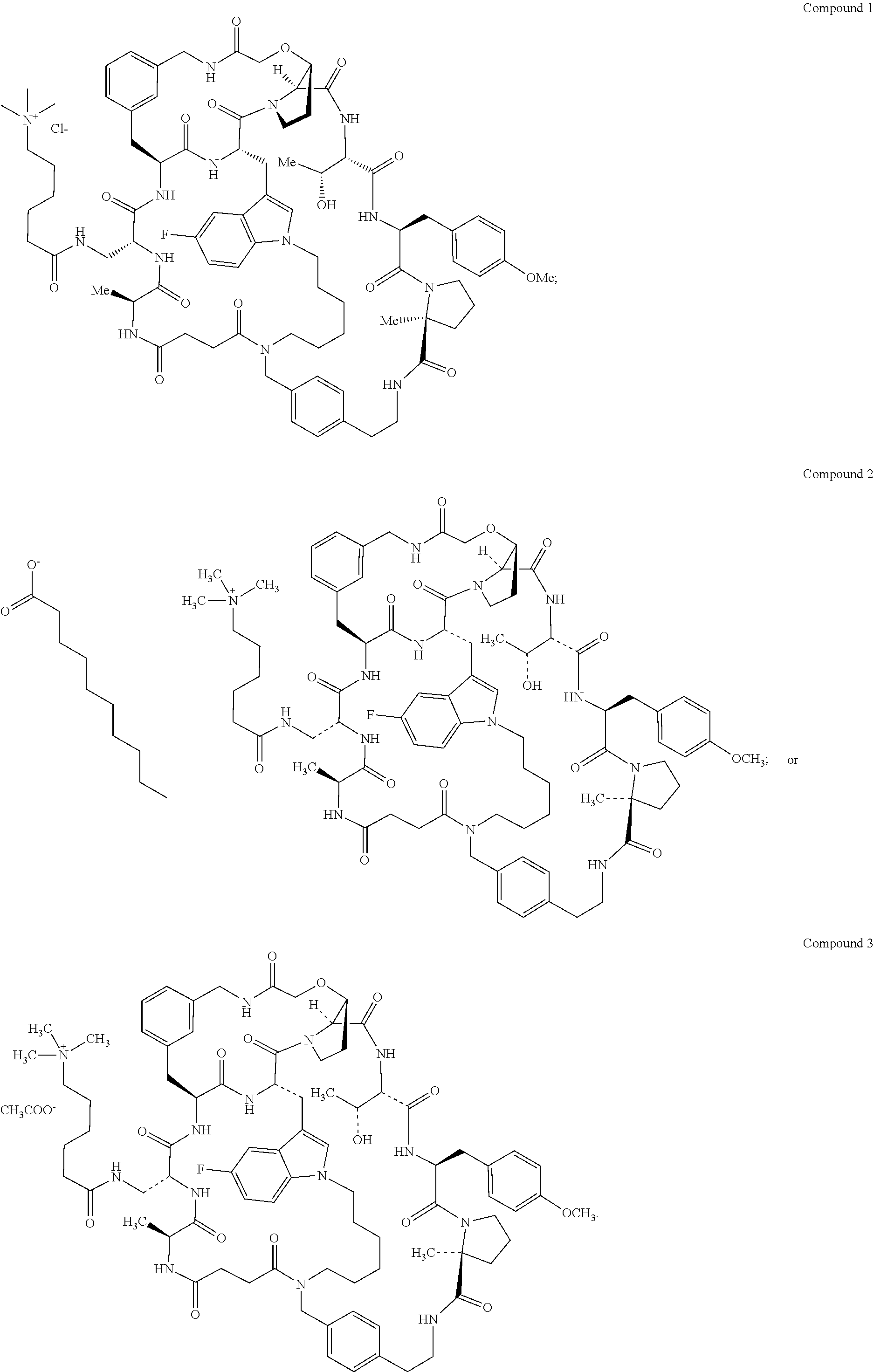

14. A method of reducing LDL-C in a subject in need thereof comprising orally administering to the subject an amount of a compound of formula (I) 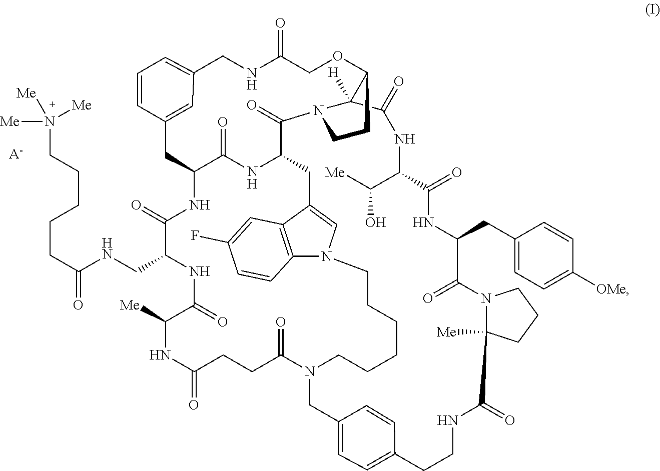
wherein A⁻ is a pharmaceutically acceptable anion selected from caprate or acetate, and wherein the amount is from about 5 mg to about 300 mg of the compound of formula (I).

15. A method of treating atherosclerotic cardiovascular disease in a subject in need thereof comprising orally administering to the subject an amount of a compound of formula (I) 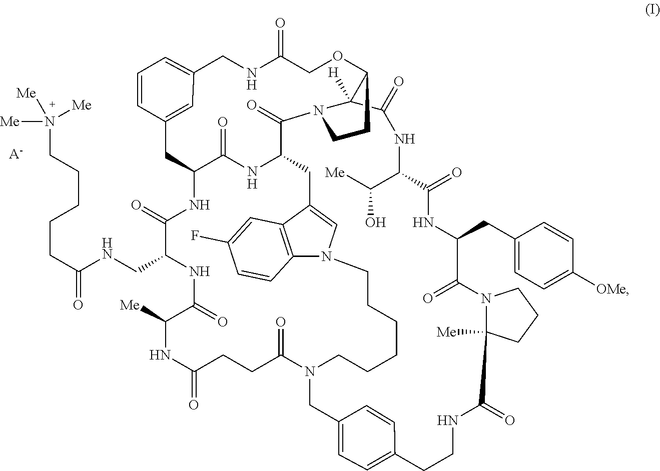
wherein A⁻ is a pharmaceutically acceptable anion selected from caprate or acetate, and wherein the amount is from about 5 mg to about 300 mg of the compound of formula (I).

16. A method of inhibiting PCSK9 activity in a subject in need of such treatment comprising orally administering to the subject an amount of a compound of formula (I) 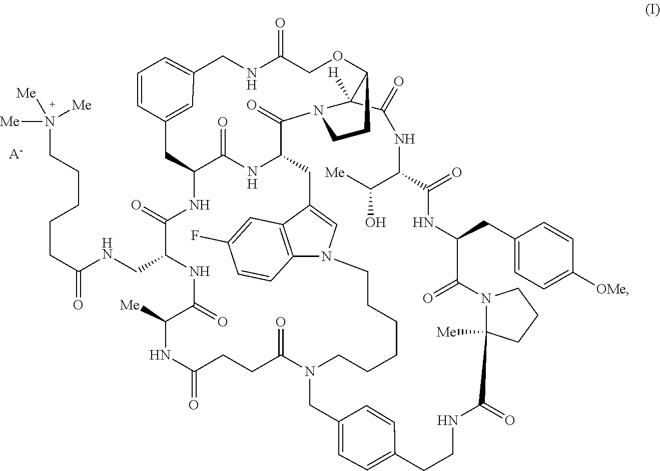
wherein A⁻ is a pharmaceutically acceptable anion selected from caprate or acetate, and wherein the amount is from about 5 mg to about 300 mg of the compound of formula (I).

17. The method according to claim 1, wherein the subject in need fasts approximately 30 minutes before the administration of a compound of formula (I).

18. A pharmaceutical composition comprising a compound of formula (I) 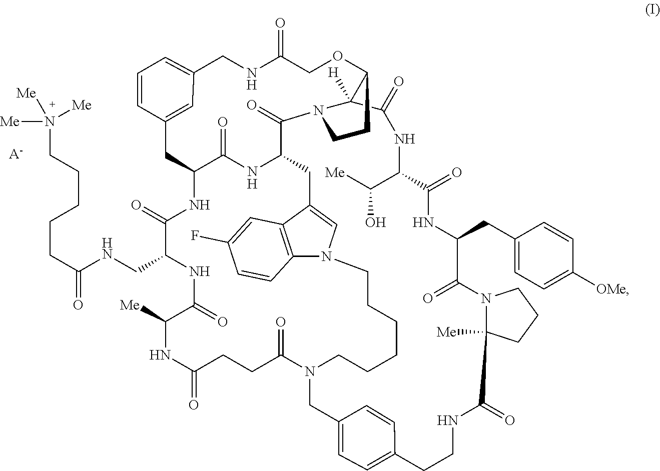
wherein A⁻ is a pharmaceutically acceptable anion, and a permeation enhancer.

19. The pharmaceutical composition of claim 18, wherein the permeation enhancer is sodium caprate.

20. The pharmaceutical composition of claim 18, further comprising a diluent selected from a polyethylene glycol, microcrystalline cellulose, mannitol, starch, dicalcium phosphate, calcium carbonate, sodium carbonate, lactose or combinations thereof.

21. The pharmaceutical composition of claim 20, wherein the diluent is selected from microcrystalline cellulose, lactose or macrogol (PEG 4000).

22. The pharmaceutical composition of claim 18, wherein the pharmaceutical composition is in the form of a tablet.

23. The pharmaceutical composition of claim 18, wherein the pharmaceutical composition is in the form of a capsule.

24. The pharmaceutical composition of claim 18, wherein the compound of formula (I) is Compound 1, Compound 2, or Compound 3.

25. The pharmaceutical composition of claim 18, wherein the pharmaceutical composition comprises a) about 1% to 7% by weight relative to the total weight of the pharmaceutical composition of a compound of formula (I); b) about 1% to 75% by weight relative to the total weight of the pharmaceutical composition of a permeation enhancer; c) at least one diluent; and d) optionally a glidant and/or a lubricant.

26. The pharmaceutical composition of claim 18, wherein the pharmaceutical composition comprises a) about 2% to 6% by weight relative to the total weight of the pharmaceutical composition of a compound of formula (I); b) about 18% to 74% by weight relative to the total weight of the pharmaceutical composition of a permeation enhancer, where the permeation enhancer is sodium caprate; c) at least one diluent selected from PEG4000, microcrystalline cellulose and lactose; d) 0% to about 3% by weight relative to the total weight of the pharmaceutical composition of a glidant, where the glidant is silicon dioxide; e) 0% to about 2% by weight relative to the total weight of the pharmaceutical composition of a lubricant where the lubricant is magnesium stearate and f) optionally at least one disintegrant.
